SEQUENCE LISTING (22 sequences)

SEQ ID NO: 1 - RNA, 18 nt, Artificial sequence
  Synthetic oligonucleotide
acgugaaaaaaaauguca

SEQ ID NO: 2 - DNA, 18 bp, Artificial sequence
  Synthetic oligonucleotide
ugacauttttttucacgu

SEQ ID NO: 3 - DNA, 18 bp, Artificial sequence
  Synthetic oligonuc. RNA and 2′-deoxy-2′-fluoro-4′-thioarabinothymidine
ugacaunnnnnnucacgu

SEQ ID NO: 4 - DNA, 18 bp, Artificial sequence
  Synthetic oligonuc. RNA and 2′-deoxy-2′-fluoroarabinothymidine
ugacaunnnnnnucacgu

SEQ ID NO: 5 - DNA, 18 bp, Artificial sequence
  Synthetic oligonucleotide
tgacattttttttcacgt

SEQ ID NO: 6 - RNA, 18 nt, Artificial sequence
  Synthetic oligonucleotide
ugacauuuuuuuucacgu

SEQ ID NO: 7 - DNA, 18 bp, Artificial sequence
  Synthetic oligonucleotide
acgtgaaaaaaaatgtca

SEQ ID NO: 8 - DNA, 21 bp, Artificial sequence
  Synthetic oligonucleotide
gcuugaagucuuuaauuaatt

SEQ ID NO: 9 - DNA, 21 bp, Artificial sequence
  Synthetic oligonucleotide
uuaauuaaagacuucaagcgg

SEQ ID NO: 10 - DNA, 21 bp, Artificial sequence
  Synthetic oligonucleotide
uuaauuaaagacuucaagcgg

SEQ ID NO: 11 - DNA, 21 bp, Artificial sequence
  Synthetic oligonucleotide
gcuugaagucuuuaannaatt

SEQ ID NO: 12 - DNA, 21 bp, Artificial sequence
  Synthetic oligonucleotide
gcuugaagucuuuaannaatt

SEQ ID NO: 13 - DNA, 21 bp, Artificial sequence
  Synthetic oligonucleotide
uuaauuaaagacnucaagcgg

SEQ ID NO: 14 - DNA, 21 bp, Artificial sequence
  Synthetic oligonucleotide
uuaauuaaagacnucaagcgg

SEQ ID NO: 15 - DNA, 21 bp, Artificial sequence
  Synthetic oligonucleotide
nnaauuaaagacuucaagcgg

SEQ ID NO: 16 - DNA, 21 bp, Artificial sequence
  Synthetic oligonucleotide
nnaauuaaagacuucaagcgg

SEQ ID NO: 17 - DNA, 21 bp, Artificial sequence
  Synthetic oligonucleotide
nnaauuaaagacuucaagcgg

SEQ ID NO: 18 - DNA, 21 bp, Artificial sequence
  Synthetic oligonucleotide
nnaauuaaagacuucaagcgg

SEQ ID NO: 19 - DNA, 21 bp, Artificial sequence
  Synthetic oligonucleotide
gcuugaagucuuuaauuaagg

SEQ ID NO: 20 - DNA, 21 bp, Artificial sequence
  Synthetic oligonucleotide
gcttgaagtctttaattaagg

SEQ ID NO: 21 - DNA, 21 bp, Artificial sequence
  Synthetic oligonucleotide
gcttgaagtctttaauuaatt

SEQ ID NO: 22 - DNA, 19 bp, Artificial sequence
  Synthetic oligonucleotide
gcttgaagtctttattaaa

(19) United States
(12) Patent Application Publication
(10) Pub. No.: US 2009/0069263 A1
(43) Pub. Date: Mar. 12, 2009

(54) 4'-THIOARABINONUCLEOTIDE-CONTAINING OLIGONUCLEOTIDES, COMPOUNDS AND METHODS FOR THEIR PREPARATION AND USES THEREOF

(76) Inventors: Masad J. Damha, Saint-Hubert (CA); Jonathan K. Watts, Montreal (CA); B. Mario Pinto, Coquitlam (CA)

Correspondence Address: GOUDREAU GAGE DUBUC, 2000 MCGILL COLLEGE, SUITE 2200, MONTREAL, QC, H3A 3H3, CA

(21) Appl. No.: 12/097,376

(22) PCT Filed: Dec. 14, 2006
(86) PCT No.: PCT/CA2006/002035

§ 371 Date: Jun. 13, 2008

Related U.S. Application Data
(60) Provisional application No. 60/750,838, filed on Dec. 16, 2005.

(51) Int. Cl. C07H 21/00; C07H 19/00; A61K 31/7088; C12N 15/11; A61K 48/00; C07H 21/02; C07F 9/6553

(52) U.S. Cl. 514/44; 536/22.1; 536/24.5; 536/25.3; 435/440; 549/6

(57) ABSTRACT

Oligonucleotides comprising one or more 4′-thioarabinonucleotides are described, as well as uses thereof for applications such as antisense- and RNAi-based gene silencing. 4′-thioarabinose-based phosphoramidite and H-phosphonate compounds are also described, as well as uses thereof for the synthesis of oligonucleotides comprising one or more 4′-thioarabinonucleotides.

### CROSS REFERENCE TO RELATED APPLICATIONS

This application claims the benefit, under 35 U.S.C. § 119(e), of U.S. provisional application Ser. No. 60/750,838 filed on Dec. 16, 2005, which is incorporated herein by reference in its entirety.

### FIELD OF THE INVENTION

The invention relates to oligonucleotides, compounds and methods for their preparation and uses thereof, such as for silencing the expression of a nucleic acid or gene of interest using small interfering RNA (siRNA) or antisense technologies (using antisense oligonucleotides [AONs]).

### BACKGROUND OF THE INVENTION

Numerous strategies for silencing gene expression with nucleic acid-based molecules are under development. Of these, the hybridization-driven “antisense” strategies, using ribozymes, DNAzymes, and antisense oligonucleotides (AONs) such as chimeric RNA-DNA (gapmers) or phosphorothioate DNA (PS-DNA) have received the greatest attention (Stephenson et al. 1978; Uhlmann et al. 1990). More recently, RNA interference (RNAi) has emerged as an exciting potential alternative to these more classical approaches (Fire et al., 1998, Elbashir et al. 2001). There are several reports describing the utility of this method for silencing genes in living organisms, ranging from yeast to mammals.

In the field of antisense therapy, one strategy developed uses “gapmer” oligonucleotides such as the following 5′-MMM MMM LLL LLL MMM MMM-3′, wherein M is a type of nucleotide that is not capable of inducing RNase-H cleavage (e.g. RNA, 2′-OMe-RNA), and L is a type of nucleotide that is capable of inducing such cleavage (e.g. DNA, 2′F-ANA).

Both these techniques present significant challenges, and there is a need for improvements in for example efficacy, in vivo stability and reduction of “off-target” effects (e.g., the silencing of a gene other than the intended target).

Furthermore, both native DNA and RNA are subject to relatively rapid degradation, mediated primarily by 3′-exonucleases, but also as a result of endonuclease attack. Thus, to obtain clinically useful molecules, it is desirable for antisense and siRNA molecules to have enhanced stability, as well as enhanced strength of hybridization with RNA (reviewed in Mangos et al. 2002). In addition, in the absence of a delivery vehicle, these molecules also need to be able to cross cell membranes and then hybridize with their intended RNA target. Also, RNA tertiary structure is a further factor which can affect the ability of antisense oligonucleotides and siRNA to hybridize with their target. It is furthermore undesirable for either type of molecule to exert non-sequence-specific binding.

There is therefore a continued need for improved oligonucleotide-based approaches.

### SUMMARY OF THE INVENTION

The invention relates to an oligonucleotide comprising a 4′-thioarabinose modified nucleotide, compounds and methods for their preparation and uses thereof, and uses thereof.

Accordingly, in a first aspect, the invention provides an oligonucleotide comprising at least one 4′-thioarabinose-modified nucleotide.

In an embodiment, the above-mentioned oligonucleotide is from about 5 to about 100 nucleotides in length, in further embodiments from about 10 to about 100, from about 5 to about 50, from about 10 to about 50, from about 15 to about 50, from about 10 to about 30, from about 18 to about 27, from about 19 to about 27, from about 18 to about 25, from about 19 to about 25, or from about 19 to about 23, nucleotides in length. In an embodiment, the above-mentioned oligonucleotide is made up of both RNA-like and DNA-like nucleotides. In an embodiment the above-mentioned oligonucleotide further comprises one or more DNA-like nucleotides. In an embodiment, the above-mentioned oligonucleotide further comprises one or more RNA-like nucleotides other than a 4′-thioarabinose-modified nucleotide.

In an embodiment, the above-mentioned oligonucleotide is capable of inducing RNase H-mediated cleavage of a complementary RNA strand.

In an embodiment, the above-mentioned oligonucleotide is 5′-phosphorylated. In the case of

In an embodiment, the above-mentioned oligonucleotide is capable of hybridizing to a complementary oligonucleotide thereby to form a double-stranded siRNA-like molecule, where the 4′-thioarabinose-modified nucleotide may be present in either one or both strands. In an embodiment, one or both strands have overhangs from 1-5 (e.g. 2 nucleotides) nucleotides on the 3′-end. In an embodiment, neither strand has an overhang. In embodiments, either or both strands comprise chemical modification(s) at one or more terminal nucleotides, such as to confer resistance to phosphorylation. In an embodiment, the overhanging nucleotides are DNA-like nucleotides (e.g. 2′-deoxyribonucleotides, 2′-deoxy-2′-fluoroarabinonucleotides or combinations thereof). In embodiments, either or both strands are phosphorylated at the 5′-end (e.g., by chemical or enzymatic phosphorylation).

In embodiments, the sense strand is modified at the 5′-end to prevent phosphorylation.

In an embodiment, the above-mentioned oligonucleotide is 15-80 nucleotides in length and comprises a first sequence and a second sequence complementary to said first sequence such that the oligonucleotide or a portion thereof is capable of adopting an siRNA-like hairpin structure in which the first and second sequences form the stem of the hairpin structure.

In an embodiment, the above-mentioned 4′-thioarabinose-modified nucleotide is present within the 5′-terminal 8 nucleotides of the oligonucleotide.

In an embodiment, the above-mentioned 4′-thioarabinose-modified nucleotide is present within the 5′-terminal 8 nucleotides, in a further embodiment, within the 5′-terminal 2 nucleotides, of either or both strands of the double-stranded siRNA-like molecule. In a further embodiment, the two 5′-terminal nucleotides are 4′-thioarabinose-modified nucleotides.

In an embodiment, the above-mentioned 4′-thioarabinose-modified nucleotide is present within the 3′-terminal 8 nucleotides of the sense strand, in a further embodiment, within the 3′-terminal 2 nucleotides, of the double-stranded siRNA-like molecule. In a further embodiment, the two 3′-terminal nucleotides are 4′-thioarabinose-modified nucleotides.

In an embodiment, one strand of the above-mentioned double-stranded siRNA-like molecule comprises the 4′-thioarabinose-modified nucleotide and the other strand comprises a 2′-deoxy-2′-fluoroarabinonucleotide. In an embodiment, the strand comprising the 4′-thioarabinose-modified nucleotide is the antisense strand of the double-stranded siRNA-like molecule.

In an embodiment, the above-mentioned arabinose modified nucleotide comprises a 2′ substituent selected from the group consisting of fluorine, hydroxyl, amino, azido, alkyl, alkoxy, and alkoxyalkyl groups. In an embodiment, the alkyl group is selected from the group consisting of methyl, ethyl, propyl, butyl, and functionalized alkyl groups. In an embodiment, the functionalized alkyl group is selected from the group consisting of as ethylamino, propylamino and butylamino groups. In an embodiment, the alkoxy group is selected from the group consisting of methoxy, ethoxy, propoxy and functionalized alkoxy groups. In an embodiment, the functionalized alkoxy group is selected from the group consisting of —O(CH₂)_{q}—R, where q=2-4 and R is —NH₂, —OCH₃, or —OCH₂CH₃. In an embodiment, the alkoxyalkyl group is selected from the group consisting of methoxyethyl, and ethoxyethyl.

In an embodiment, the above-mentioned 4′-thioarabinose modified nucleotide is a 2′-deoxy-2′-fluoro-4′-thioarabinonucleotide (2′F-4′S-ANA).

In an embodiment, the above-mentioned oligonucleotide comprises two or more types of arabinose-modified nucleotides. In an embodiment, the two or more types of arabinose-modified nucleotides are present in the same strand, different strands or both strands of the double-stranded siRNA-like molecule. In embodiments, the two or more types of arabinose modified nucleotides are 2′-deoxy-2′-fluoro-4′-thioarabinonucleotide (2′F-4′S-ANA) and 2′-deoxy-2′-fluoro-arabinonucleotide (2′F-ANA).

In an embodiment, the above-mentioned oligonucleotide has a sugar phosphate backbone.

In an embodiment, the above-mentioned oligonucleotide comprises at least one internucleotide linkage selected from the group consisting of phosphodiester, phosphotriester, phosphorothioate, methylphosphonate, boranophosphate and any combination thereof.

In an embodiment, the above-mentioned oligonucleotide comprises heterocyclic canonical bases selected from the group consisting of Adenine, Cytosine, Guanine, Thymine and Uracil.

In an embodiment, the above-mentioned oligonucleotide comprises a modified (non-canonical) base.

In an embodiment, the ends of the above-mentioned oligonucleotide are capped with modified nucleotides or moieties capable of conferring exonuclease resistance.

In a further aspect, the invention provides a siRNA or siRNA-like molecule comprising the above-mentioned oligonucleotide.

In a further aspect, the invention provides a double-stranded siRNA or siRNA-like molecule comprising (a) a first oligonucleotide comprising the above-mentioned oligonucleotide of the invention and (b) a second oligonucleotide complementary thereto. In a further embodiment, the second oligonucleotide comprises the above-mentioned oligonucleotide of the invention.

In embodiments, the first and second oligonucleotides are 19 to 23 nucleotides in length. In an embodiment, the double-stranded siRNA or siRNA-like molecule comprises a 19-21 bp duplex portion. In an embodiment, the double-stranded siRNA or siRNA-like molecule comprises a 1-5 (e.g. 2 nucleotide) nucleotide 3′ overhang in one or both strands.

In a further aspect, the invention provides a method for increasing therapeutic efficacy, nuclease stability, and/or selectivity of binding of an oligonucleotide, the method comprising replacing at least one nucleotide of the oligonucleotide with a 4′-thioarabinose modified nucleotide and/or incorporating a 4′-thioarabinose modified nucleotide into the oligonucleotide. In an embodiment, the 4′-thioarabinose modified nucleotide is a 2′-deoxy-2′-fluoro-4′-thioarabinonucleotide (2′F-4′S-ANA).

In a further aspect, the invention provides a pharmaceutical composition comprising the above-mentioned oligonucleotide and a pharmaceutically acceptable carrier.

In a further aspect, the invention provides a use of the above-mentioned oligonucleotide, siRNA or siRNA-like molecule or composition for gene silencing.

In a further aspect, the invention provides a use of the above-mentioned oligonucleotide or siRNA or siRNA-like molecule for the preparation of a medicament.

In a further aspect, the invention provides a use of the above-mentioned oligonucleotide or siRNA or siRNA-like molecule for the preparation of a medicament for gene silencing.

In a further aspect, the invention provides a method of inhibiting gene expression in a biological system, comprising introducing into the system the above-mentioned oligonucleotide, siRNA or siRNA-like molecule or composition.

In a further aspect, the invention provides a method of inhibiting gene expression in a subject, comprising administering a therapeutically effective amount of the above-mentioned oligonucleotide, siRNA or siRNA-like molecule or composition to the subject.

In a further aspect, the invention provides a method of treating a condition associated with expression of a gene in a subject, the method comprising administering the above-mentioned oligonucleotide, siRNA or siRNA-like molecule or composition to the subject, wherein the oligonucleotide is targeted to the gene.

In a further aspect, the invention provides a kit or commercial package comprising: (i) the above-mentioned oligonucleotide; (ii) the above-mentioned oligonucleotide and a second oligonucleotide complementary thereto; (iii) the above-mentioned siRNA or siRNA-like molecule; or (iv) the above-mentioned composition; together with instructions for use of any of (i) to (iv) for: (a) gene silencing; (b) inhibiting gene expression in a biological system; (c) inhibiting gene expression in a subject; (d) treating a condition associated with expression of a gene in a subject; or (e) any combination of (a) to (d).

In a further aspect, the invention provides a method of preparing the above-mentioned oligonucleotide comprising incorporating at least one 4′-thioarabinose-modified nucleotide monomer during oligonucleotide synthesis.

According to an aspect of the invention, nucleic acid oligomers containing at least one 4′-thioarabinose modified nucleotide are provided. In an embodiment, the 4′-thioarabinose modified nucleotide is a 2′-deoxy-2′-fluoro-4′-thioarabinose modified nucleotide (2′F-4′S-ANA).

Up to now, 2′-fluoroarabinonucleotide derivatives (4′-oxygen) have been known to exhibit a well known “DNA-like” conformation (Trempe et al. 2001). Very surprisingly, it was determined in the studies described herein that 4′-thio-modified arabinose nucleotides adopt an “RNA-like” conformation. Because of this very particular RNA-like conformation, it is shown herein that oligonucleotides comprising one or more of such monomers adopt an RNA-like conformation and in turn RNA-like activity and function. Thus, oligonucleotides containing one or more 4′-thioarabinonucleotide derivatives are useful as RNA-based gene silencing reagents when used via antisense and RNAi methodologies. “DNA-like” as used herein in reference to conformation refers to a conformation of for example a modified nucleoside or nucleotide which is similar to the conformation of a corresponding unmodified DNA unit. DNA-like conformation may be expressed for example as having a southern P value (see FIG. 4 and Example 3). “RNA-like” as used herein in reference to conformation refers to a conformation of for example a modified nucleoside or nucleotide which is similar to the conformation of a corresponding unmodified RNA unit. RNA-like conformation may be expressed for example as having a northern P value (see FIG. 4 and Example 3). Further, RNA-like molecules tend to adopt an A-form helix while DNA-like molecules tend to adopt a B-form helix.

In a further aspect of the invention, oligonucleotides 15-50 nucleotides in length are modified with at least one 2′F-4′S-ANA unit.

In a further aspect of the invention, a double-stranded RNA oligonucleotide is provided, where one or both strands may be modified with at least one 4′-thioarabinose modified nucleotide, for example:

| | |
|---|---|
| Sense | 5′-NNN NNN NNN NNN NNN Nnn-3′ |
| Antisense | 3′-nnN NNN NNN NNN NNN NNN-5′ |

where N represents RNA, DNA or 2′F-4′S-ANA nucleotides (or combinations thereof), and n are overhanging RNA, DNA or 2′F-4′S-ANA nucleotides on the 3′-end of one or both strands. Alternatively, the duplex may have one or two blunt ends.

In an embodiment, the above duplex is a hairpin duplex, that is a single strand which is self-complementary and folds back onto itself.

In a further embodiment, a single-stranded oligonucleotide chimera is provided which is composed of M and intervening L residues, e.g.,

[M]x-[L]y-[M]x

in which:
M represents 2′F-4′S-ANA, or combinations of 2′-modified-RNA and 2′F-4′S-ANA; the 2′-modified RNA is chosen from 2′F-RNA, 2′-O-alkyl-RNA, RNA and a combination thereof.
L represents DNA-like modifications that elicit RNase H activity such as DNA, arabinonucleotides (ANA), 2′-deoxy-2′-fluoroarabinonucleotides (2′F-ANA), cyclohexene nucleic acids (CeNA) and alpha-L-locked nucleic acids (α-L-LNA) and combinations thereof.

In embodiments, the internucleotide linkages are phosphodiesters, phosphorothioates or combination thereof.

In other embodiments of the invention, the 2′-F substituent of the 2′F-4′S-ANA residue may be substituted with a group selected from the group consisting of 2′-hydroxyl, 2′-amino, 2′-azido, 2′-alkyl, 2′-alkoxy, and 2′-alkoxyalkyl groups. In a further embodiment of the invention, the 2′-alkyl group is selected from the group consisting of methyl, ethyl, propyl, butyl, and functionalized alkyl groups such as cyanoethyl, ethylamino, propylamino and butylamino groups. In embodiments, the alkoxy group is selected from the group consisting of 2′-methoxy, 2′-ethoxy, 2′-proproxy and functionalized alkoxy groups such as 2′-O(CH₂)_{q}—R, where q=2-4 and —R is a —NH₂, —OCH₃, or —OCH₂CH₃ group. In embodiments, the 2′-alkoxyalkyl group is selected from the group consisting of methoxyethyl, and ethoxyethyl.

In other embodiments of the invention, the oligonucleotide (or, in the case of a double-stranded oligonucleotide, either strand) is fully substituted with 2′F-4′S-ANA (sF) modified nucleotides, giving a strand [sF]x, typically x=4 to 30 nt. The heterocyclic base moiety of any nucleotides in the oligonucleotide AON and RNAi constructs described may be one of the canonical bases of DNA or RNA, for example, adenine, cytosine, guanine, thymine or uracil. In other embodiments of the invention, some of the heterocyclic base moieties may be made up of modified or non-canonical bases, for example, inosine, 5-methylcytosine, 2-thiothymine, 4-thiothymine, 7-deazaadenine, 9-deazaadenine, 3-deazaadenine, 7-deazaguanine, 9-deazaguanine, 6-thioguanine, isoguanine, 2,6-diaminopurine, hypoxanthine, and 6-thiohypoxanthine.

In other embodiments of the invention, the oligonucleotide comprises one or more internucleotide linkages selected from the group consisting of:

a) phosphodiester;
b) phosphotriester;
c) phosphorothioate;
d) methylphosphonate; and
e) boranophosphate.

According to another aspect of the invention, a method for increasing at least one of therapeutic efficacy, nuclease stability, or selective binding of an oligonucleotide (or, in the case of a double-stranded oligonucleotide, either strand) is provided. The method comprises replacing at least one nucleotide of the oligonucleotide (or, in the case of a double-stranded oligonucleotide, either strand) with a corresponding number of 4′-thioarabinose modified nucleotides.

According to another aspect of the invention, a method of inhibiting a deleterious gene (“gene silencing”) in a patient in need thereof is provided. “Gene silencing” as used herein refers to an inhibition or reduction of the expression of the protein encoded by a particular nucleic acid sequence or gene (e.g., a deleterious gene). The method comprises administering to the patient a therapeutically effective amount of the pharmaceutical composition of the invention.

According to another aspect of the invention a pharmaceutical composition is provided, comprising the oligonucleotide (or, in the case of a double-stranded oligonucleotide, either strand) of the present invention along with a pharmaceutically acceptable carrier.

According to another aspect of the invention a commercial package is provided. The commercial package comprises the oligonucleotide or pharmaceutical composition of the present invention together with instructions for its use for inhibiting gene expression.

In a further aspect, the invention provides a compound of the Formula I, described herein. In a further aspect, the invention provides a compound of the Formula III, described herein. In a further aspect, the invention provides a compound of the Formula V, described herein, or a salt thereof. In a further aspect, the invention provides a compound of the Formula VI, described herein.

In a further aspect, the invention provides a method of preparing a compound of Formula I, III, V or VI described herein, the method comprising phosphitylation of a compound of Formula VI described herein.

In a further aspect, the invention provides a method of synthesizing the above-mentioned oligonucleotide, the method comprising: (a) 5′-deblocking; (b) coupling; (c) capping; and (d) oxidation; wherein (a), (b), (c) and (d) are repeated under conditions suitable for the synthesis of the oligonucleotide, and wherein the synthesis is carried out in the presence of a phosphoramidite or H-phosphonate monomer base comprising the compound of the Formula I, III, V or VI described herein. In an embodiment, a phosphoramidite or H-phosphonate monomer base other than the compound the compound of the Formula I, III, V or VI is also incorporated into the oligonucleotide during its synthesis.

In a further aspect, the invention provides a kit comprising the compound of the Formula I, III, V, VI or combinations thereof together with instructions for its use in oligonucleotide synthesis.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The invention will now be described in greater detail having regard to the appended drawings in which:

FIG. 1 illustrates schematically the synthesis of 2′-deoxy-2′-fluoro-5-methyl-4′-thioarabinouridine. Reagents and conditions: (a) Li, liq. NH₃, −78° C.; (b) TIPSCl₂, pyridine, rt, 3 h; (c) DAST, CH₂Cl₂, −15° C., 15 min; (d) Bu₄NF, THF, rt, 30 min; (e) BzCl, pyridine, rt, 6 h; (f) O₃, CH₂Cl₂, −78° C., 30 min; (g) Ac₂O, 110° C., 3 h; (h) bis-silylated thymine, TMSOTf, CCl₄, reflux, 16 h, 47% yield of β product; (i) 2M NH₃ in MeOH, rt, 23 h, 87%.

FIG. 2 illustrates the 3′-O-benzoate participation in the glycosylation reaction. Increased participation occurs in nonpolar solvents in which the thiacarbenium ion is less stable.

FIG. 3 illustrates schematically the synthesis of the 2′-deoxy-2′-fluoro-5-methyl-4′-thioarabinouridine 3′-O-phosphoramidite. Reagents and conditions: (a) 2M NH₃ in MeOH, rt, 23 h; (b) DMTrCl, Pyridine, rt, 44 h; (c) (N(ⁱPr₂))₂P(OCH₂CH₂CN), Diisopropylammonium tetrazolide, CH₂Cl₂, rt, 68 h.

FIGS. 4a and 4b illustrate the pseudorotational wheel describing the conformations of nucleosides, along with examples of significant nucleoside conformations. (a) The pseudorotational wheel describing the conformations of nucleosides; E=envelope, T=twist. (b) Examples of significant nucleoside conformations for DNA (X═H) and 2′F-ANA (X═F).

FIG. 5 provides definitions of internal torsion angles in a nucleoside.

FIGS. 6 to 15 illustrate torsion angle graphs used to obtain Aⱼ and Bⱼ. FIG. 6: Aⱼ and Bⱼ for H1′-H2′ coupling in FMAU; FIG. 7: Aⱼ and Bⱼ for H1′-F2′ coupling in FMAU; FIG. 8: Aⱼ and Bⱼ for H2′-H3′ coupling in FMAU; FIG. 9: Aⱼ and Bⱼ for F2′-H3′ coupling in FMAU; FIG. 10: Aⱼ and Bⱼ for H3′-H4′ coupling in FMAU; FIG. 11: Aⱼ and Bⱼ for H1′-H2′ coupling in 4′S-FMAU; FIG. 12: Aⱼ and Bⱼ for H1′-F2′ coupling in 4′S-FMAU; FIG. 13: Aⱼ and Bⱼ for H2′-H3′ coupling in 4′S-FMAU; FIG. 14: Aⱼ and Bⱼ for F2′-H3′ coupling in 4′S-FMAU; FIG. 15: Aⱼ and Bⱼ for H3′-H4′ coupling in 4′S-FMAU.

FIG. 16 shows circular dichroism spectra (a: I-V, ssRNA target; b: I-V, ssDNA target). Spectra were run at 20° C. after annealing the duplexes under the same conditions described for the binding studies.

FIG. 17 shows a Ribonuclease H (RNase H) degradation of various hybrid duplexes. An 18-nt 5′-³²P-labeled target RNA (5′-ACG UGA AAA AAA AUG UCA-3′; [SEQ ID NO:1]) was preincubated with complementary 18-nt I-V, and then added to reaction assays containing either (a) E. coli RNase HI or (b) human RNase HII (110 nM assay shown here). Aliquots were removed as listed on diagrams (in minutes). Base sequences of antisense oligomers are given in Table 7.

FIG. 18 shows the activity of 2′F-4′S-ANA-modified siRNA, and compares with 2′F-ANA modifications at the same positions (sequences given in Table 8). For each duplex tested, the values shown from left to right for each group correspond to the following concentrations, respectively: 40 nM, 10 nM, 2 nM, 0.4 nM, 0.08 nM, 0.016 nM, and 0.0032 nM.

FIG. 19 shows RNA interference data demonstrating the effect of phosphorylation on siRNAs modified at the 5′-terminal of the antisense strand (sequences given in Table 8). For each duplex tested, the values shown from left to right for each group correspond to the following concentrations, respectively: 40 nM, 10 nM, 2 nM, 0.4 nM, 0.08 nM, 0.016 nM, and 0.0032 nM.

FIG. 20 shows the activity of 2′F-4′S-ANA in combination with various heavily-modified sense strands (sequences given in Table 9). For each duplex tested, the values shown from left to right for each group correspond to the following concentrations, respectively: 40 nM, 10 nM, 2 nM, 0.4 nM, 0.08 nM, 0.016 nM, and 0.0032 nM.

### DETAILED DESCRIPTION OF THE INVENTION

The invention relates to oligonucleotides containing 4′-thioarabinose modified nucleotides and compounds which may be used for their preparation. These modifications are shown herein to be RNA mimics and therefore are useful in various types of RNA-based technologies, such as gene silencing approaches. The invention further relates to 4′-thioarabinose nucleoside 3′-O-phosphoramidite or 3′-O-H-phosphonate compounds, which may be used for example for the preparation of an oligonucleotide of the invention.

As shown in the Examples below, the 2′-deoxy-2′-fluoro-4′-thioarabinose modification is shown herein to adopt an RNA-like conformation in nucleosides, by conformational analysis using NMR coupling constants and the program PSEUROT. This finding is of great significance because the conformation of oligonucleotides is believed to depend strongly upon the conformation of the nucleotide monomers that make them up.

It is further shown herein that the 2′-deoxy-2′-fluoro-4′-thioarabinose modification binds to complementary RNA with an affinity very similar to that of unmodified RNA, by UV thermal denaturation studies. Having an affinity similar to that of RNA allows both efficient, selective binding and high turnover rates in for example antisense or siRNA applications.

### Antisense Applications

In embodiments, the invention provides oligonucleotides of the invention and uses thereof as antisense molecules for exogenous administration to effect the degradation and/or inhibition of the translation of a target mRNA. Examples of therapeutic antisense oligonucleotide applications, incorporated herein by reference, include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166,195 issued Nov. 24, 1992; U.S. Pat. No. 5,004,810, issued Apr. 2, 1991; U.S. Pat. No. 5,194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to the target mRNA to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target mRNA for antisense binding may include not only the information to encode a protein, but also associated ribonucleotides, which for example form the 5′-untranslated region, the 3′-untranslated region, the 5′ cap region and intron/exon junction ribonucleotides.

Oligonucleotides of the invention (e.g., antisense molecules) may include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with formacetal (O—CH₂—O), CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). Oligonucleotides having morpholino backbone structures may also be used (U.S. Pat. No. 5,034,506). In alternative embodiments, antisense oligonucleotides may have a peptide nucleic acid (PNA, sometimes referred to as “protein nucleic acid”) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al. 1991 and U.S. Pat. No. 5,539,082). The phosphodiester bonds may be substituted with structures which are chiral and enantiomerically specific. Persons of ordinary skill in the art will be able to select other linkages for use in practice of the invention.

Oligonucleotides of the invention may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙNH₂ or O(CH₂) n CH₃ where n is from 1 to about 10; C₁ to C₁₀ lower alkyl, substituted lower alkyl, alkaryl or aralkyl; Cl; Br; CN; CF₃; OCF₃; O—, S—, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

In some embodiments, the oligonucleotides (e.g., antisense oligonucleotides) in accordance with this invention may comprise from about 5 to about 100 nucleotide units, in further embodiments from about 10 to about 100, from about 5 to about 30, from about 10 to about 30, from about 18 to about 27, from about 19 to about 27, from about 18 to about 25, from about 19 to about 25, or from about 19 to about 23 nucleotide units. As will be appreciated, a nucleotide unit is a base-sugar combination (or a combination of analogous structures) suitably bound to an adjacent nucleotide unit through phosphodiester or other bonds forming a backbone structure.

siRNA (RNAi) Applications

In further embodiments, the invention provides oligonucleotides of the invention and uses thereof in siRNA/RNAi applications, whereby expression of a nucleic acid encoding a polypeptide of interest, or a fragment thereof, may be inhibited or prevented using RNA interference (RNAi) technology, a type of post-transcriptional gene silencing. RNAi may be used to create a pseudo “knockout”, i.e., a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example published US patent applications 20020173478 (Gewirtz; published Nov. 21, 2002) and 20020132788 (Lewis et al.; published Nov. 7, 2002), all of which are herein incorporated by reference. Reagents and kits for performing RNAi are available commercially from for example Ambion Inc. (Austin, Tex., USA), New England Biolabs Inc. (Beverly, Mass., USA) and Invitrogen (Carlsbad, Calif., USA).

The initial agent for RNAi in some systems is thought to be dsRNA molecule corresponding to a target nucleic acid. The dsRNA is then thought to be cleaved into short interfering RNAs (siRNAs) which are for example 21-23 nucleotides in length (19-21 bp duplexes, each with 2 nucleotide 3′ overhangs). The enzyme thought to effect this first cleavage step (the Drosophila version is referred to as “Dicer”) is categorized as a member of the RNase III family of dsRNA-specific ribonucleases. Alternatively, RNAi may be effected via directly introducing into the cell, or generating within the cell by introducing into the cell an siRNA or siRNA-like molecule or a suitable precursor (e.g. vector encoding precursor(s), etc.) thereof. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may be effected by the introduction of suitable in vitro synthesized siRNA or siRNA-like molecules into cells. RNAi may for example be performed using chemically-synthesized RNA. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods have been described (see e.g., Brummelkamp et al. [2002] Science 296:550). Various methods for introducing such vectors into cells, either in vitro or in vivo (e.g. gene therapy) are known in the art.

Accordingly, in an embodiment of the invention, a nucleic acid, encoding a polypeptide of interest, or a fragment thereof, may be inhibited by introducing into or generating within a cell an siRNA or siRNA-like molecule based on an oligonucleotide of the invention, corresponding to a nucleic acid encoding a polypeptide of interest, or a fragment thereof, or to an nucleic acid homologous thereto (sometimes collectively referred to herein as a “target nucleic acid/gene”). “siRNA-like molecule” refers to a nucleic acid molecule similar to an siRNA (e.g. in size and structure) and capable of eliciting siRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 19-23 nucleotides in length. In an embodiment, siRNA or siRNA-like molecule comprises a 19-21 bp duplex portion, each strand having a 2 nucleotide 3′ overhang. In other embodiments, one or both strands may have blunt ends. In embodiments, the siRNA or siRNA-like molecule is substantially identical to a nucleic acid encoding a polypeptide of interest, or a fragment or variant (or a fragment of a variant) thereof. Such a variant is capable of encoding a protein having activity similar to the polypeptide of interest. In embodiments, the sense strand of the siRNA or siRNA-like molecule is substantially identical to a target gene/sequence, or a fragment thereof (where, in embodiments, U may replace the T residues of the DNA sequence).

Accordingly, the invention further provides an siRNA or siRNA-like molecule comprising an oligonucleotide of the invention. In embodiments, the invention provides a double-stranded siRNA or siRNA-like molecule comprising a first oligonucleotide which is an oligonucleotide of the invention (i.e., comprising at least one 4′-thioarabinose-modified nucleotide) and a second oligonucleotide complementary thereto. In further embodiments, the invention provides a kit or package comprising a first oligonucleotide which is an oligonucleotide of the invention and a second oligonucleotide complementary thereto. In embodiments, the second oligonucleotide is also an oligonucleotide of the invention (i.e., comprising at least one 4′-thioarabinose-modified nucleotide). In embodiments, the first and second oligonucleotides are 19-23 nucleotides in length. In embodiments, the double-stranded siRNA or siRNA-like molecule comprises a 19-21 bp duplex portion. In embodiments, the double-stranded siRNA or siRNA-like molecule comprises a 3′ overhang of 1-5 nucleotides in each strand. In further embodiments, neither strand of the double-stranded siRNA or siRNA-like molecule has an overhang. In a further embodiment, the double-stranded siRNA or siRNA-like molecule comprises one or both blunt ends.

The invention further provides a method of inhibiting gene expression in a biological system, comprising introducing into the system the siRNA or siRNA-like molecule.

The invention further provides a method of inhibiting gene expression in a subject, comprising administering the siRNA or siRNA-like molecule to the subject.

The invention further provides a method of treating a condition associated with expression of a gene in a subject, the method comprising administering the siRNA or siRNA-like molecule to the subject, wherein the siRNA or siRNA-like molecule is targeted to the gene.

The invention further provides a use of the siRNA or siRNA-like molecule for the preparation of a medicament.

The invention further provides a use of the siRNA or siRNA-like molecule for a method selected from: (a) gene silencing; (b) inhibiting gene expression in a biological system; (c) inhibiting gene expression in a subject; and (d) treating a condition associated with expression of a gene in a subject; and (e) preparation of a medicament for treating a condition associated with expression of a gene in a subject.

In one of the proposed applications of 4′-thioarabinose-modified oligonucleotides, a single-stranded chimeric oligonucleotide is presented. One or more sections of this oligonucleotide are made up of RNA-like nucleotides (M) that do not elicit RNase H activity when duplexed to complementary RNA. One or more sections of this oligonucleotide are made up of DNA-like nucleotides (L) that are capable of eliciting RNase H activity when duplexed to complementary RNA.

In another application, siRNA duplexes will be partially or completely modified with the 2′F-4′S-ANA modification to provide nuclease stability reduce off-target effects while retaining strong gene silencing by virtue of the unexpected RNA-like structure of the 2′F-4′S-ANA.

In various embodiments, an oligonucleotide of the invention may be used therapeutically in formulations or medicaments to prevent or treat disease associated with the expression of a target nucleic acid or gene. The invention provides corresponding methods of medical treatment, in which a therapeutic dose of an oligonucleotide of the invention is administered in a pharmacologically acceptable formulation, e.g. to a patient or subject in need thereof. Accordingly, the invention also provides therapeutic compositions comprising an oligonucleotide of the invention and a pharmacologically acceptable excipient or carrier. In one embodiment, such compositions include an oligonucleotide of the invention in a therapeutically or prophylactically effective amount sufficient to treat a disease associated with the expression of a target nucleic acid or gene. The therapeutic composition may be soluble in an aqueous solution at a physiologically acceptable pH.

A “therapeutically effective amount” refers to an amount effective, at dosages and for periods of time necessary, to achieve the desired therapeutic result, such as a reduction or reversal in progression of a disease associated with the expression of a target nucleic acid or gene. A therapeutically effective amount of an oligonucleotide of the invention may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of onset or progression of a disease associated with the expression of a target nucleic acid or gene. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like, that are physiologically compatible. In one embodiment, the carrier is suitable for parenteral administration. Alternatively, the carrier can be suitable for intravenous, intraperitoneal, intramuscular, topical, sublingual or oral administration, or for administration by inhalation. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, an oligonucleotide of the invention can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. an oligonucleotide of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, an oligonucleotide of the invention may be formulated with one or more additional compounds that enhance its solubility.

In accordance with another aspect of the invention, therapeutic compositions of the present invention, comprising an oligonucleotide of the invention, may be provided in containers or commercial packages which further comprise instructions for its use for the inhibition of target gene expression, and/or prevention and/or treatment of a disease associated with expression of a target nucleic acid or gene.

Accordingly, the invention further provides a commercial package comprising an oligonucleotide of the invention or the above-mentioned composition together with instructions for inhibition of expression of a target nucleic acid or gene or for the prevention and/or treatment of a disease associated with expression of a target nucleic acid or gene.

The invention further provides a use of an oligonucleotide of the invention or the above-mentioned composition for inhibition of expression of a target nucleic acid or gene or for the prevention and/or treatment of a disease associated with expression of a target nucleic acid or gene. The invention further provides a use of an oligonucleotide of the invention for the preparation of a medicament for prevention and/or treatment of a disease associated with expression of a target nucleic acid or gene.

“Nucleoside” refers to a base-sugar combination, the base being attached to the sugar via an N-glycosidic linkage. “Nucleotide” refers to a nucleoside that additionally comprises a phosphate group attached to the sugar portion of the nucleoside. “Base”, “nucleic acid base” or “nucleobase” refer to a heterocyclic base moiety, which within a nucleoside or nucleotide is attached to the sugar portion thereof, generally at the 1′ position of the sugar moiety. This term includes both naturally-occurring and modified bases. The two most common classes of naturally-occurring bases are purines and pyrimidines, and comprise for example guanine, cytosine, thymine, adenine and uracil. A number of other naturally-occurring bases, as well as modified bases, are known in the art, for example, inosine, 5-methylcytosine, 2-thiothymine, 4-thiothymine, 7-deazaadenine, 9-deazaadenine, 3-deazaadenine, 7-deazaguanine, 9-deazaguanine, 6-thioguanine, isoguanine, 2,6-diaminopurine, hypoxanthine, and 6-thiohypoxanthine.

The invention further provides a compound of the Formula I:

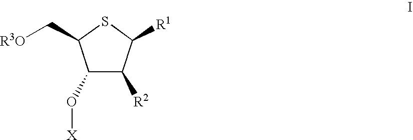

wherein:

R¹ is a canonical or modified nucleobase;

R² is selected from the group consisting of a halogen, OH, and alkoxy;

R³ is a protecting group; and

X is selected from the group consisting of a phosphoramidite moiety and an H-phosphonate moiety.

In embodiments, R² is a halogen selected from the group consisting of F and Cl.

In embodiments, R² is OMe (methoxy).

In a further embodiment, X is a linker moiety capable of attachment to or covalently attached to a solid support.

In an embodiment, the protecting group R³ is selected from the group consisting of monomethoxytrityl, dimethoxytrityl, levulinyl, and silyl-based protecting groups.

In an embodiment X is a phosphoramidite moiety of the Formula II:

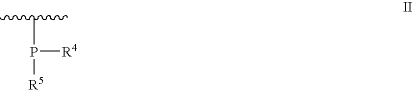

wherein:

R⁴ is a dialkylamino group NR⁹R¹⁰, wherein R⁹ and R¹⁰ are each independently lower alkyl groups, linear or branched; and

R⁵ is a substituted or unsubstituted alkoxy group OR¹¹, wherein R¹¹ is selected from the group consisting of methyl, beta-cyanoethyl, p-nitrophenylethyl, trimethylsilylethyl, or other linear or branched alkyl or functionalized alkyl groups. The central phosphorous atom has a lone pair of electrons and is thus trivalent.

Accordingly, the invention further provides a compound of the Formula III:

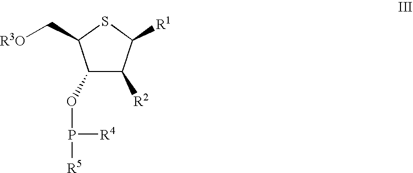

wherein R¹-R⁵ and R⁹-R¹¹ are as defined above.

In an embodiment, X is an H-phosphonate moiety of the Formula IV:

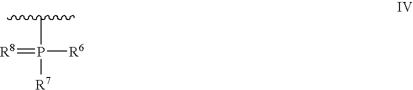

wherein:

R⁶ is H;

R⁷ is selected from the group consisting of OH and an oxyanion (O—) associated or paired with a cationic ion (e.g. a trialkylammonium ion, e.g. a triethylammonium ion); and

R⁸ is selected from the group consisting of O and S.

Accordingly, the invention further provides a compound of the Formula V or a salt thereof:

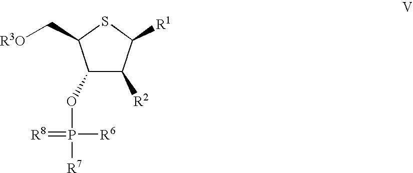

wherein R¹-R³ and R⁶-R⁸ are as defined above.

The invention further provides a compound of the Formula VI:

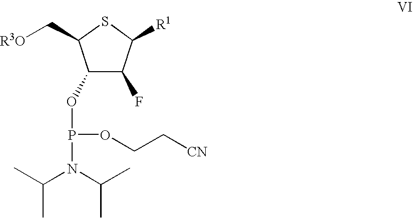

wherein R¹ and R³ are as defined above.

The invention further provides a method of preparing the above-mentioned compound of the Formula I, III, V or VI, the method comprising: (a) providing a 4′-thioarabinonucleoside compound of the Formula VII:

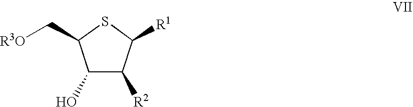

wherein R¹, R² and R³ are as defined above, and wherein if R′ is a base selected from the group consisting of adenine, guanine and cytosine, the amino group thereof comprises an attached protecting group; and

(b) phosphitylation of the 3′-hydroxyl group of the compound of the Formula VII.

In embodiments, the method further comprises protection of the 5′-hydroxyl group of the 4′-thioarabinonucleoside thereby to generate —OR³ at the 5′ position (R³ as defined above), and/or protection of the amino group of the heterocyclic base in the case where R¹ is selected from Adenine, Guanine and Cytosine, prior to the phosphitylation of the 3′-hydroxyl group.

In embodiments, protection of the heterocyclic base may comprise the transformation of the exocyclic amines of Ade, Gua and Cyt bases into amides or other groups stable to the conditions of solid-phase oligonucleotide synthesis. In an embodiment, protection of the heterocyclic base may involve transformation of the exocyclic amines of Ade and Cyt into benzamide groups, and the exocyclic amine of G into an isobutyramide. Alternatively, the exocyclic amines of the nucleobases may be protected as N-PAC (N-phenoxyacetyl) derivatives. In embodiments, the protection of the exocyclic amines may be achieved by reaction with the corresponding acyl chloride, or another reactive acyl derivative.

In embodiments, protection of the 5′-hydroxyl group involves the addition of a group stable to the conditions of coupling, capping and oxidation during oligonucleotide synthesis, but able to be selectively and quantitatively removed after each step. In embodiments, protection of the 5′-hydroxyl group may involve reaction with a chloride, including an aryl chloride, alkoxyaryl chloride, or silyl chloride, to produce an ether. In a further embodiment, protection of the 5′-hydroxyl group may involve reaction with dimethoxytrityl chloride or monomethoxytrityl chloride, to yield the corresponding 5′-O-trityl ether. In a further embodiment, protection of the 5′-hydroxyl group may involve reaction with an activated acyl compound, for example levulinic anhydride or levulinyl chloride, to produce the corresponding ester (e.g., 5′-O-levulinyl ester).

In an embodiment, phosphitylation of the 3′-hydroxyl group, involves a chlorophosphoramidite, where the two other groups attached to phosphorus are as defined above as R⁴ and R⁵. In other embodiments, the activated phosphoramidite is a phosphorodiamidite, containing two amino groups defined above as R⁴, and one R⁵. In this case the phosphorodiamidite is reacted with a weak acid, capable of activating only the first of the R⁴ groups to yield the desired nucleoside phosphoramidite as defined above. In another embodiment, the phosphitylation of the 3′-hydroxyl group involves a reaction with a phosphitylating agent, such as PX₃ (X=e.g. 1,2,4-triazolide), followed by addition of water, to provide an H-phosphonate group.

The invention further provides a kit comprising the above-mentioned 4′-thioarabinonucleoside compound (e.g., a compound of Formula VII), or a precursor thereof lacking a 5′ protecting group and/or protecting group on the amino group of the heterocyclic base in the case where the base is Adenine, Guanine or Cytosine, together with instructions for its use to prepare a compound of the Formula I, III, V or VI. In embodiments, such a kit may further comprise one or more further reagents which may be used in carrying out the method, such as those used in, phosphitylation, 5′-protection, protection of an amino group of a heterocyclic base, or combinations thereof. In embodiments, the kit comprises the above-mentioned 4′-thioarabinonucleoside compound or precursor thereof corresponding to each of the canonical bases A, C, G, T and U, or subsets thereof (such as [A, C, G and U] or [A, C, G and T]).

The invention further provides a method of synthesizing an oligonucleotide of the invention, the method comprising: (a) 5′-deblocking; (b) coupling; (c) capping; and (d) oxidation; wherein (a), (b), (c) and (d) are repeated under conditions suitable for the synthesis of the oligonucleotide, wherein the synthesis is carried out in the presence of a nucleoside phosphoramidite or H-phosphonate monomer comprising a compound of Formula I, III, V or VI described herein or combinations thereof. In embodiments, a nucleoside phosphoramidite or H-phosphonate monomer other than the compound of Formula I, III, V or VI described herein may be additionally utilized and incorporated into the oligonucleotide during such synthesis.

In embodiments, the synthesis is carried out on a solid phase, such as on a solid support selected from the group consisting of controlled pore glass, polystyrene, polyethylene glycol, polyvinyl, silica gel, silicon-based chips, cellulose paper, polyamide/kieselgur and polacryloylmorpholide. In further embodiments, the monomers may be used for solution phase synthesis or ionic-liquid based synthesis of oligonucleotides.

“Protecting group” as used herein refers to a moiety that is temporarily attached to a reactive chemical group to prevent the synthesis of undesired products during one or more stages of synthesis. Such a protecting group may then be removed to allow for step of the desired synthesis to proceed, or to generate the desired synthetic product. Examples of protecting groups are trityl (e.g., monomethoxytrityl, dimethoxytrityl), silyl, levulinyl and acetyl groups.

“5′-Deblocking” as used herein refers to a step in oligonucleotide synthesis wherein a protecting group is removed from a previously added nucleoside (or a chemical group linked to a solid support), to produce a reactive hydroxyl which is capable of reacting with a nucleoside molecule, such as a nucleoside phosphoramidite or H-phosphonate.

“Coupling” as used herein refers to a step in oligonucleotide synthesis wherein a nucleoside is covalently attached to the terminal nucleoside residue of the oligonucleotide (or to the solid support via for example a suitable linker), for example via nucleophilic attack of an activated nucleoside phosphoramidite, H-phosphonate, phosphotriester, pyrophosphate, or phosphate in solution by a terminal 5′-hydroxyl group of a nucleotide or oligonucleotide bound to a support. Such activation may be effected by an activating reagent such as tetrazole, 5-ethylthio-tetrazole, 4,5-dicyanoimidazole (DCI), and/or pivaloyl chloride.

“Capping” as used herein refers to a step in oligonucleotide synthesis wherein a chemical moiety is covalently attached to any free or unreacted hydroxyl groups on the support bound nucleic acid or oligonucleotide (or on a chemical linker attached to the support). Such capping is used to prevent the formation of for example sequences of shorter length than the desired sequence (e.g., containing deletions). An example of a reagent which may be used for such capping is acetic anhydride. Further, the capping step may be performed either before or after the oxidation (see below) of the phosphite bond.

“Oxidation” as used herein refers to a step in oligonucleotide synthesis wherein the newly synthesized phosphite triester or H-phosphonate diester bond is converted into pentavalent phosphate triester or diester bond. In the case where a phosphorothioate internucleotide linkage is desired, “oxidation” also refers to the addition of a sulfur atom to generate a phosphorothioate linkage.

“Alkyl” as used herein refers to the radical of saturated aliphatic groups, including straight chain (linear) alkyl groups, branched-chain alkyl groups, cycloalkyl (alicyclic) groups, alkyl substituted cycloalkyl groups, and cycloalkyl substituted alkyl groups. Typical alkyl groups include, but are not limited to, methyl, ethyl, propyl, isopropyl, butyl, isobutyl, t-butyl, pentyl, isopentyl, hexyl, etc. “Lower alkyl” groups can be (C₁-C₆) alkyl, in a further embodiment (C₁-C₃) alkyl. A “substituted alkyl” has substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

“Alkenyl” and “alkynyl” refer to unsaturated aliphatic groups analogous in length and possible substitution to the alkyls described above, but that contain at least one double or triple bond respectively. An “alkenyl” is an unsaturated branched, straight chain, or cyclic hydrocarbon radical with at least one carbon-carbon double bond. The radical can be in either the cis or trans conformation about the double bond(s). Typical alkenyl groups include, but are not limited to, ethenyl, propenyl, isopropenyl, butenyl, isobutenyl, tert-butenyl, pentenyl, hexenyl, etc. An “alkynyl” is an unsaturated branched, straight chain, or cyclic hydrocarbon radical with at least one carbon-carbon triple bond. Typical alkynyl groups include, but are not limited to, ethynyl, propynyl, butynyl, isobutynyl, pentynyl, hexynyl, etc.

The invention further provides a kit comprising the above-mentioned compound (e.g., a compound of Formula I, III, V or VI described herein) together with instructions for its use in oligonucleotide synthesis. In embodiments, such a kit may further comprise one or more further reagents which may be used in carrying out the method, such as those used in the 5′-deblocking, coupling, capping and oxidation steps mentioned above, or combinations thereof. In a further embodiment, the kit may further comprise a phosphoramidite or H-phosphonate monomer base other than the compound of Formula I, III, V or VI described herein. In an embodiment, the kit comprises versions of the above-mentioned compound (e.g., a compound of Formula I, III, V or VI described herein) corresponding to each of the canonical bases A, C, G, T and U, or subsets thereof (such as [A, C, G and U] or [A, C, G and T]).

The invention further provides a salt of any of the above-mentioned compounds where applicable.

The following examples are illustrative of various aspects of the invention, and do not limit the broad aspects of the invention as disclosed herein.

### EXAMPLES

### Example 1

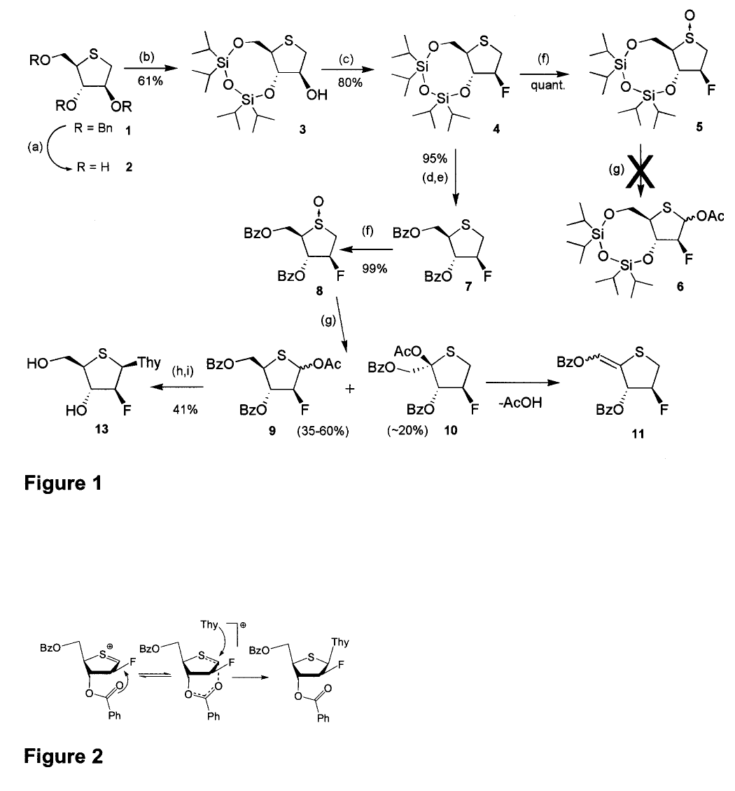

### Chemical Synthesis of 2′-deoxy-2′-fluoro-4′-thioarabinonucleotides (FIGS. 1 & 2)

2,3,5-Tri-O-benzyl-1,4-anhydro-4-thio-arabinitol (1) was prepared from L-xylose following a procedure similar to that of Satoh et al (Satoh et al. 1998). The benzyl protecting groups were removed by Birch reduction using Li/liq. NH₃ to give the triol 2. Treatment of the triol 2 with equimolar ratios of 1,3-dichloro-1,1,3,3-tetraisopropyldisiloxane (TIPSCl₂) (Naka et al. 2000) in pyridine gave mainly the desired compound 3 which, when treated with DAST, gave within 10 min the desired 2-fluoro derivative 4 in 80% yield. Moreover, the reaction proceeded with retention of configuration, presumably through an episulfonium ion intermediate (Yuasa et al. 1990).

In order to install the pyrimidine base at C-1, we chose to functionalize C-1 as an acetate derivative through the Pummerer reaction, as reported by Naka et al (Naka et al. 2000). The thioether 4 was thus subjected to ozonization at −78° C. to give the sulfoxide 5 quantitatively. When compound 5 was treated with Ac₂O at 70° C., several components were observed on TLC, suggesting that the silyl protecting group was being removed. We therefore decided to replace the 3,5-O-disiloxane bridge with benzoyl protecting groups. Thus, the thioether 4 was treated with Bu₄NF followed by BzCl in pyridine to give compound 7 in excellent yield. Ozonization of thioether 7 at −78° C. afforded the sulfoxide 8 which, when treated with Ac₂O at 110° C., gave mainly the desired 1-O-acetyl derivative 9 as an anomeric mixture (α:β 1:2 to 1:14). The minor isomer, the 4-O-acetate 10, was found to undergo spontaneous elimination of acetic acid to yield the exocyclic olefin 11 over a period of several weeks at room temperature (FIG. 1).

N-Glycosylation of acetate derivative 9 was next accomplished by coupling to thymine in the presence of TMS-trifluoromethanesulfonate as the Lewis acid catalyst (FIG. 1). We propose that the α-face of the molecule is partially blocked by a benzoxonium ion resulting from attack of the benzoate ester on the thiacarbenium ion (FIG. 2), as has been observed using other 3′-directing groups (Young et al. 1994). This mechanism would be more favored in nonpolar solvents where a localized cation is highly unstable (Table 1). Accordingly, our use of nonpolar solvents improves the β:α ratio significantly over that reported in the literature for similar Lewis acid-catalyzed glycosylations (Yoshimura et al. 2000). The α nucleoside 12α was removed by silica gel column chromatography. Debenzoylation of 12β using 2 M methanolic ammonia gave 13 in 87% yield.

Details of synthetic methods and characterization of compounds follow:

1,4-Anhydro-3,5-O— (1,1,3,3-tetraisopropyldisiloxane-1,3-diyl)-4-thio-D-arabinitol (3). To a solution of 1,4-anhydro-4-thio-D-arabinitol 2 (2.10 g, 14.0 mmol) in anhydrous pyridine (10 ml) was added 1,3-dichloro-1,1,3,3-tetraisopropyldisiloxane (5.30 g, 16.8 mmol). The reaction mixture was stirred at room temperature for 3 h and then quenched by addition of ice. The mixture was concentrated under reduced pressure and the resultant brown syrup was dissolved in ethyl acetate (30 ml) and washed with ice cold 1% aqueous HCl (3×15 ml), followed by brine. The organic layer was dried (Na₂SO₄), concentrated and the residue was purified by column chromatography (eluent 30% EtOAc/Hex) to give 3 (3.38 g, 8.61 mmol, 61%) as an oil. [α]_{D}:−4 (c 1.2, CH₂Cl₂); ¹H NMR (400 MHz, CDCl₃): δ 4.17 (br ddd, 1H, ³J_{1a,2}=6.7, ³J_{1b,2}=8.8, ³J_{2,3}=7.8 Hz, H-2), 4.02 (dd, 1H, ³J_{3,4}=7.9, H-3), 3.99 (dd, 1H, ³J_{4,5a}=3.2, ²J_{5a,5b}=12.3 Hz, H-5a), 3.78 (dd, 1H, ³J_{4,5b}=5.8 Hz, H-5b), 3.24 (ddd, 1H, H-4), 2.95 (dd, 1H, ²J_{1a,1b}=10.4 Hz, H-1a), 2.73 (dd, 1H, H-1b), 2.20 (br s, 1H, OH), 1.20-0.90 (m, 28H, 4×SiCH(CH₃)₂); ¹³C NMR (100.61 MHz, CDCl₃): δ 79.9 (C-2), 77.6 (C-3), 63.0 (C-5), 48.6 (C-4), 30.5 (C-1), 17.4, 17.3, 17.2, 17.1, 17.0 (CH₃), 13.6, 13.3, 12.8, 12.7 (SiCH); MALDI MS: m/e 415.13 (M⁺+Na). Anal. Calcd for C₁₇H₃₆O₄SSi₂: C, 51.99; H, 9.24. Found: C, 51.83; H, 9.32.

1,4-Anhydro-2-deoxy-2-fluoro-3,5-O— (1,1,3,3-tetraisopropyldisiloxane-1,3-diyl)-4-thio-D-arabinitol (4). A solution of DAST (1.2 g, 9.5 mmol) in anhydrous CH₂Cl₂ (5 ml) was added dropwise to a solution of 3 (3.1 g, 7.9 mmol) in anhydrous CH₂Cl₂ (15 mL) with cooling to −20° C. After 15 min at −20° C., the reaction mixture was quenched by addition of ice, and the mixture was partitioned between CH₂Cl₂ and water. The separated organic layer was washed with saturated NaHCO₃ followed by brine. The organic layer was dried (Na₂SO₄), concentrated and the residue was purified by column chromatography, eluted with 15% EtOAc in hexane, to give 4 (2.5 g, 6.3 mmol, 80%) as a colorless oil. [α]_{D}:−14 (c 1.5, CH₂Cl₂); ¹H NMR (400 MHz, CDCl₃): δ 4.98 (dddd, 1H, ²J_{2,F}=53.4, J_{1b,2}=7.8, ³J_{2,3}=7.3, ³J_{1a,2}=6.9 Hz, H-2), 4.34 (ddd, 1H, ³J_{3,F}=15.3, ³J_{3,4}=8.2 Hz, H-3), 3.99 (ddd, 1H, ²J_{5a,5b}=12.3, ³J_{4,5a}=3.3, ⁵J_{5a,F}=0.5 Hz, H-5a), 3.79 (ddd, 1H, ³J_{4,5b}=5.6, ⁵J_{5b,F}=1.9 Hz, H-5b), 3.21 (dddd, 1H, ⁴J_{4,F}=1.1 Hz, H-4), 3.05 (ddd, 1H, ²J_{1a,1b}=11.1, ³J_{1a,F}=6.9 Hz, H-1a), 2.90 (ddd, 1H, ³J_{1b,F}=16.9 Hz, H-1b), 1.30-0.85 (m, 28H, 4×SiCH(CH₃)₂); ¹³C NMR (100.61 MHz, CDCl₃): δ 96.6 (d, ¹J_{2,F}=189.2 Hz, C-2), 77.8 (d, ²J_{3,F}=22.9 Hz, C-3), 62.5 (C-5), 47.6 (d, ³J_{4,F}=7.6 Hz, C-4), 28.7 (d, ²F_{1,F}=22.1 Hz, C-1), 17.5, 17.4, 17.3, 17.1, 17.0 (CH₃), 13.7, 13.4, 13.1, 12.9 (SiCH). MALDI MS: m/e 374.94 (M⁺-F). Anal. Calcd for C₁₇H₃₅FO₃SSi₂: C, 51.73; H, 8.94. Found: C, 51.76; H, 8.93.

1,4-Anhydro-2-deoxy-2-fluoro-3,5-di-O-benzoyl-4-thio-D-arabinitol (7). To a solution of 4 (2.46 g, 6.23 mmol) in THF (10 ml) was added a 1M solution of tetra-n-butylammonium fluoride in THF (3.0 mL, 3 mmol). The reaction mixture was stirred at room temperature for 30 min. The reaction mixture was concentrated on a rotary evaporator with bath temperature below 30° C. The crude syrup was dissolved in ethyl acetate (50 ml) and washed with small volumes of water and brine. The organic layer was dried over anhydrous Na₂SO₄ and concentrated to yield crude 1,4-anhydro-2-deoxy-2-fluoro-4-thio-D-arabinitol as a pale-yellow syrup.

This crude diol (1.05 g) was redissolved in anhydrous pyridine (10 mL) and the mixture was cooled in an ice bath before adding benzoyl chloride (4.0 mL, 34 mmol). The reaction mixture was stirred at room temperature for 6 h and then was quenched by addition of ice. The mixture was concentrated under vacuum and the resultant brown syrup was dissolved in ethyl acetate (30 mL) and washed with ice cold 1% aqueous HCl (3×15 ml), followed by brine. The organic layer was dried (Na₂SO₄), concentrated, and the residue was purified by column chromatography, eluted with 30% EtOAc in hexane, to give 7 (2.14 g, 5.94 mmol, 95%) as an oil. [α]_{D}:+41 (c 0.78, CHCl₃); ¹H NMR (400 MHz, CDCl₃): δ 8.05 (m, 4H, Ar), 7.62 (m, 2H, Ar), 7.44 (m, 4H, Ar), 5.84 (ddd, 1H, ³J_{3,F}=9.6, ³J_{2,3}=2.6, ³J_{3,4}=2.6 Hz, H-3), 5.38 (dddd, 1H, ²J_{2,F}=49.4, ³J_{1a,2}=4.3, ³J_{1b,2}=2.9 Hz, H-2), 4.55 (ddd, ²J_{5,5b}=11.1, ³J_{4,5a}=7.1, ⁵J_{5a,F}=1.7 Hz, H-5a), 4.49 (dd, 1H, ³J_{4,5b}=8.5, ⁵J_{5b,F}<1 Hz, H-5b), 3.88 (br dd, 1H, ⁴J_{4,F}<1 Hz, H-4), 3.36 (ddd, 1H, ³J_{1a,F}=30.5, ²J_{1a,1b}=12.6 Hz, H-1a), 3.31 (ddd, 1H, ³J_{1b,F}=19.3 Hz, H-1b); ¹³C NMR (100.61 MHz, CDCl₃): δ 166.0, 164.9 (C═O), 133.6, 133.1 (Ar), 129.8, 129.7 (Ar), 128.5, 128.4 (Ar), 96.2 (d, ¹J_{2,F}=183.1 Hz, C-2), 78.9 (d, ²J_{3,F}=28.9 Hz, C-3), 65.5 (d, ⁴J_{5,F}=4.5 Hz, C-5), 48.9 (C-4), 34.6 (d, ²J_{1,F}=22.8 Hz, C-1). MALDI MS: m/e 383.20 (M⁺+Na). Anal. Calcd for C₁₉H₁₇FO₄S: C, 63.32; H, 4.75. Found: C, 63.60; H, 4.80.

1,4-Anhydro-2-deoxy-2-fluoro-3,5-di-O-benzoyl-4-sulfinyl-D-arabinitol (8). Ozone gas was bubbled through a clear solution of 7 (2.10 g, 5.83 mmol) in CH₂Cl₂ (15 mL) at −78° C. The reaction was complete in 30 min, as indicated by persistence of a blue color. Nitrogen gas was bubbled through the solution to remove excess ozone until the blue color vanished. The reaction mixture was allowed to warm to room temperature and concentrated under reduced pressure. The residue was purified by column chromatography, eluted with 30% EtOAc in hexane, to give 8 (2.18 g, 5.79 mmol, 99%) as a white solid. Mp. 141-142° C. Spectral data for the α-isomer: ¹H NMR (500 MHz, CDCl₃): δ 8.05 (m, 4H, Ar), 7.60 (m, 2H, Ar), 7.45 (m, 4H, Ar), 5.80 (dddd, 1H, ²J_{2,F}=49.6, ³J_{1a,2}=5.3, ³J_{1b,2}=4.8. ³J_{2,3}=3.9 Hz, H-2), 5.74 (ddd, 1H, ³J_{3,F}=13.2, ³J_{3,4}=3.9 Hz, H-3), 4.89 (dd, ²J_{5a,5b}=11.9, ³J_{4,5a}=4.9, ⁵J_{5a,F}˜0 Hz, H-5a), 4.74 (dd, 1H, ³J_{4,5b}=7.5, ⁵J_{5b,F}˜0 Hz, H-5b), 3.65 (ddd, 1H, ⁴J_{4,F}˜0 Hz, H-4), 3.75 (ddd, 1H, ³J_{1a,F}=14.9, ²F_{1a,1b}=14.1 Hz, H-1a), 3.45 (ddd, 1H, ³J_{1b,F}=25.7 Hz, H-1b); ¹³C NMR (100.61 MHz, CDCl₃): δ 165.7, 165.3 (C═O), 134.0, 133.5, 130.1, 129.7, 128.6, 128.5 (Ar) 95.44 (d, ¹J_{2,F}=184.6 Hz, C-2), 77.3 (d, ²J_{3,F}=29.0 Hz, C-3), 71.6 (C-4), 61.1 (d, ⁴J_{5,F}=3.0 Hz, C-5), 55.8 (d, ²J_{1,F}=19.8 Hz, C-1) MALDI MS: m/e 377.20 (M⁺+H), 399.15 (M⁺+Na). Anal. Calcd for C₁₉H₁₇FO₅S: C, 60.63; H, 4.55. Found: C, 60.79; H, 4.53.

1-O-Acetyl-2-deoxy-2-fluoro-3,5-di-O-benzoyl-4-thio-α/β-D-arabinofuranose (9). A mixture of 8 (2.15 g, 2.38 mmol) and Ac₂O (6.0 ml) were heated at 110° C. for 3 h. The reaction was quenched by addition of ice after cooling to room temperature. The mixture was partitioned between EtOAc (10 mL) and water (10 mL) and further stirred for 2 h at ambient temperature. The separated organic layer was washed with saturated aqueous NaHCO₃, followed by brine. The organic layer was dried (Na₂SO₄) and concentrated in vacuo. The crude colorless oil was purified by column chromatography, eluted with 5% EtOAc in hexane, to give a mixture of the α,β-anomers of 9 (1.90 g, 4.54 mmol, 35-60%) as a white solid (β:α=2.3:1). Recrystallization (Hex:EtOAc) allowed separation of 9β: Mp. 141-142° C. (lit. (Yoshimura et al. 1999) 85-93° on mixture of anomers.) The major by-product was the 4′-acetate 10 (˜20%) which was removed by chromatography.

NMR data for major isomer 9β: ¹H NMR (400 MHz, CDCl₃): δ 8.10-7.90 (m, 4H, Ar), 7.60-7.25 (m, 6H, Ar), 6.17 (d, 1H, ³J_{1,2}=4.4, ³J_{1,F}˜0 Hz, H-1a), 6.08 (ddd, 1H, ³J_{3,F}=11.7, ³J_{3,4}=7.3, ³J_{2,3}=9.0 Hz, H-3), 5.31 (ddd, 1H, ²J_{2,F}=50.7 Hz, H-2), 4.68 (dd, ²J_{5a,5b}=11.4, ³J_{4,a}=6.1, ⁵J_{5a,F}˜0 Hz, H-5a), 4.49 (ddd, 1H, ³J_{4,5b}=6.4, ⁵J_{5b,F}=0.5 Hz, H-5b), 3.74 (ddd, 1H, ⁴J_{4,F}=6.3 Hz, H-4), 2.12 (s, 3H, CH₃); ¹³C NMR (100.61 MHz, CDCl₃): δ 169.66 (COCH₃), 165.84, 165.41 (COPh), 133.62, 133.19 (Ar) 129.86, 129.72, 128.51, 128.29 (Ar), 92.44 (d, ¹J_{2,F}=206.8 Hz, C-2), 75.7 (d, ²J_{3,F}=22.9 Hz, C-3), 73.9 (d, ²J_{1,F}=16.8 Hz, C-1), 66.0 (C-5), 42.4 (d, ³J_{4,F}=6.9 Hz, C-4), 21.0 (CH₃). ¹⁹F NMR (282.3 MHz, CDCl₃): δ −191.75 (dd, J=9 Hz, 51 Hz). The anomeric assignment was done on the basis of the vanishingly small coupling between H-1 and F.

NMR data for minor isomer 9α: ¹H NMR (400 MHz, CDCl₃): δ 8.10-7.90 (m, 4H, Ar), 7.60-7.25 (m, 6H, Ar), 6.23 (ddd, 1H, ³J_{1,F}=13.9, ³F_{1,2}=2.2, ⁴J_{1,4}=0.7 Hz, H-1a), 5.88 (ddd, 1H, ³J_{3,F}=12.3, ³J_{3,4}=3.5, ³J_{2,3}=3.7 Hz, H-3), 5.39 (ddd, 1H, ²J_{2,F}=47.6 Hz, H-2), 4.55 (dd, ²J_{5a,5b}=11.4, ³J_{4,5a}=7.8, ⁵J_{5a,F}=0.6 Hz, H-5a), 4.47 (ddd, 1H, ³J_{4,5b}=6.6, ⁵J_{5b,F}=1.5 Hz, H-5b), 4.10 (ddd, 1H, ⁴J_{4,F}=4.4 Hz, H-4), 2.12 (s, 3H, CH₃); ¹³C NMR (100.61 MHz, CDCl₃): δ 169.44 (COCH₃), 165.89, 164.98 (COPh), 133.70, 133.19 (Ar) 129.71, 129.35, 128.85, 128.37 (Ar), 98.44 (d, ²J_{2,F}=187.7 Hz, C-2), 81.47 (d, ³J_{1,F}=32.8 Hz, C-1), 77.5 (C-3), 64.9 (C-5), 49.2 (C-4), 20.8 (CH₃); ¹⁹F NMR (282.3 MHz, CDCl₃) δ −186.98 (ddd, J=12 Hz, 12 Hz, 48 Hz). The ¹H NMR data for both isomers is essentially identical to that already reported for an unassigned mixture of anomers (Yoshimura et al. 1999). For the α,β mixture: MALDI MS: m/e 441.25 (M⁺+Na); 457.18 (M⁺+Ka). Anal. Calcd for C₂₁H₁₉FO₆S: C, 60.28; H, 4.58. Found: C, 60.45; H, 4.60.

Characterization of (2S,3S,4S)-2-Acetoxy-3-benzoyloxy-2-benzoyloxymethyl-4-fluorotetrahydrothiophene (10). ¹H NMR (400.13 MHz, CDCl₃): δ 7.96, 7.85 (2 d, 4H, meta of OBz), 7.52, 7.37 (2 m, 6H, ortho and para of OBz), 6.19 (dd, 1H, J_{H3-H2}=4.3 Hz, J_{H3-F}=9.4 Hz, H-3), 5.29 (dddd, J_{H2-F}=50 Hz, J_{H2-H1}≈J_{H2-H1}=4.4 Hz, H-2), 5.25, 4.66 (2 d, 2H, J_{H5-H5′}=12.0 Hz, H-5, H-5′), 3.37 (m, 2H, H-1, H-1′), 2.12 (s, 3H, CH₃). ¹³C NMR (100 MHz, CDCl₃): δ 169.5, 165.4, 164.5 (3 C═O), 134-128.5 (aromatic), 94.0 (C-4), 93.8 (d, J_{C2-F}=190 Hz, C-2), 80.1 (d, J_{C3-F}=26.2 Hz, C-3), 64.2 (C-5), 34.4 (d, J_{C1-F}=22.3 Hz, C-1), 22.2 (CH₃). ESI-MS calcd. for C₂₁H₁₇FO₆S+Na: 441.08, found, 441.0. Stereochemistry was assigned and the structure confirmed by X-ray crystallography (data not shown.)

Characterization of (3S,4S)-2-Benzoyloxy-1-benzoyloxymethylenyl-3-fluorotetrahydrothiophene (11). ¹H NMR (400.13 MHz, CDCl₃): δ 8.10 (dd, 2H, meta of one OBz), 8.04 (s, 1H H-5), 8.00 (dd, 2H, meta of other OBz), 7.58, 7.45 (2 m, 6H, ortho and para of OBz), 6.17 (dd, 1H, J_{H3-H2}=1.6 Hz, J_{H3-F}=8 Hz, H-3), 5.35 (ddd, J_{H2-F}=48 Hz, J_{H2-H1}=3.2 Hz, H-2), 3.62 (ddd, 1H, J_{H1-F}=36 Hz, J_{H1-H1′}=13 Hz, H-1), 3.45 (dd, 1H, J_{H1′-F}=18 Hz, H-1′). NOESY crosspeaks were observed between H-3 and H-5, suggesting the Z-alkene. ¹³C NMR (100 MHz, CDCl₃): δ 165.0, 162.5 (2 OBz), 134.0, 133.8, 133.2 (2 para C and C-5), 130.3-128.6 (6 signals; meta, ortho and ipso C), 121.8 (C-4), 93.8 (d, J_{C2-F}=180 Hz, C-2), 77.8 (d, J_{C3-F}=31 Hz, C-3), 37.0 (d, J_{C1-F}=30 Hz, C-1). ¹⁹F NMR (282.3 MHz, CDCl₃): δ −185.85 (dddd, J=8, 18, 36, 48 Hz). ESI-MS Calcd for C₁₉H₁₅FO₄S+Na: 381.06; Found, 380.9.

1-(3,5-Di-O-benzoyl-2-deoxy-2-fluoro-4-thio-α/β-D-arabinofuranosyl)-thymine (12α/β). To anhydrous thymine (85 mg, 0.67 mmol) in a 25-mL round-bottomed flask was added acetonitrile (4 mL) followed by HMDS (200 μL, 153 mg, 0.95 mmol), with stirring. The mixture was heated to reflux, and became clear. After 4 h, the solvent was removed. A solution of 1-O-acetyl-2-deoxy-2-fluoro-3,5-di-O-benzoyl-4-thio-D-arabinofuranose (9, 64 mg, 0.15 mmol) in carbon tetrachloride (8 mL) was added followed by TMS-triflate (60 μL, 69 mg, 0.29 mmol). The flask that had contained the dry sugar was then rinsed with another 2-mL aliquot of carbon tetrachloride. The reaction was stirred at reflux for 16 h and monitored by TLC. It was then diluted with 15 mL CH₂Cl₂ and washed with 20 mL 5% aq. NaHCO₃. The aqueous layer was washed with 2×15 mL CH₂Cl₂. Combined organic layers were washed with 15 mL brine. The aqueous layer was washed with 10 mL CH₂Cl₂. Organic layers were dried on MgSO₄, concentrated, and purified on a silica gel column using chloroform as eluent. This system allowed partial separation of the two anomers. Compound 12β eluted first (34 mg, 47%) and was concentrated to yield an amorphous solid:

¹H NMR (400 MHz, CDCl₃): δ 8.10, 8.04 (2d, 4H, meta of OBz), 7.70 (s, 1H, H-6), 7.60 (q, 2H, para of OBz), 7.47 (d, 4H, ortho H of 2 OBz), 6.80 (dd, 1H, J_{H1′-F2′}=25.2 Hz, J_{H1′-H2′}=3.8 Hz, H-1′), 5.86 (ddd, 1H, J_{H3′-F2′}=9.4 Hz, J_{H3′-H2′}=1.8 Hz, J_{H3′-H4′}˜1 Hz, H-3′), 5.26 (ddd, 1H, J_{H2′-F2′}=49.2 Hz, H-2′), 4.69 (m, 2H, H-5′, H-5″), 4.00 (dd, 1H, J_{H4′-H5′}=J_{H4′-H5″}=7.8 Hz, H-4′), 1.94 (s, 3H, CH₃ on C5). Two pairs of NOESY crosspeaks (H6-H3′, H6-H5′) demonstrate the presence of top-face thymine and therefore the β nucleoside.

Anomer 12α was also characterized: ¹H NMR (400 MHz, CDCl₃): δ 8.04, 7.91 (2d, 4H, meta of OBz), 7.62 (s, 1H, H-6), 7.59 (q, 2H, para of OBz), 7.42 (d, 4H, ortho H of 2 OBz), 6.38 (dd, 1H, J_{H1′-F2′}=16.0 Hz, J_{H1′-H2′}=3.1 Hz, H1′), 5.81 (ddd, 1H, J_{H3′-F2′}=12.0 Hz, J_{H3′-H2′}=J_{H3′-H4′}=4.0 Hz, H3′), 5.36 (ddd, 1H, J_{H2′-F2′}=47.8 Hz, H-2′), 4.53 (m, 2H, H-5′, H-5″), 4.24 (ddd, 1H, J_{H4′-H5′}˜J_{H4′-H5″}=6.8 Hz, H-4′), 1.86 (s, 3H, CH₃ on C5). NOESY crosspeaks (H6-H2′, H6-H4′) confirmed the α configuration.

1-(2-Deoxy-2-fluoro-4-thio-β-D-arabinofuranosyl)-thymine (13). To 331 mg (0.68 mmol) of compound 12β in a round-bottomed flask equipped with a magnetic stir bar was added a 2M solution of ammonia in cold methanol (50 mL, 100 mmol). The reaction was capped with a rubber septum and allowed to stir for 23 h. It was then evaporated to dryness, adsorbed onto silica and loaded onto a short column of silica gel. Dichloromethane containing 0-3% methanol was used to elute compound 13 which was concentrated to yield an amorphous solid (164 mg, 87%):

¹H NMR (400 MHz, D₂O): δ 8.03 (s, 1H, H6), 6.09 (dd, 1H, J_{H1′-H2′}=6.0 Hz, J_{H1′-F2′}=7.9 Hz, H1′), 4.93 (ddd, 1H, J_{H2′-F2′}=50.3 Hz, J_{H2′-H3′}=7.1 Hz, H2′), 4.17 (ddd, 1H, J_{H3′-H4′}=7.0 Hz, J_{H3′-F2′}=12.1 Hz, H3′), 3.70 (m, 2H, H5′, H5″), 3.17 (ddd, 1H, J_{H4′-H5′}≈J_{H4′-H5″}=4.3 Hz, H4′), 1.68 (s, 3H, CH₃).

¹³C NMR (125 MHz, methanol-d₄): δ 165.0, 151.8 (C2, C4), 138.9 (d, J_{F2′-C6}=1.6 Hz, C6), 109.8 (C5), 96.3 (d, J_{F2′-C2′}=194.5 Hz, C2′), 73.2 (d, J_{F2′-C3′}=22.9 Hz, C3′), 60.8 (d, J_{F2′-C5′}=2.3 Hz, C5′), 58.4 (d, J_{F2-C1′}=16.8 Hz, C1′), 51.1 (d, J_{F2′-C4′}=4.6 Hz, C4′), 11.4 (CH₃).

FAB-HRMS: Calcd for C₁₀H₁₃N₂O₄SF+H⁺: 277.0658; Found: 277.0659.

The uracil congener was prepared analogously, as follows:

3′,5′-Di-O-benzoyl-2′-deoxy-2′-fluoro-4′-thio-β-D-arabinouridine (17, analogous to 12β but with uracil instead of thymine as a base moiety). To anhydrous uracil (33 mg, 0.29 mmol, 4 eq) in a 10-mL round-bottomed flask was added acetonitrile (2 mL) followed by HMDS (62 μL, 0.29 mmol, 4 eq.), with stirring. The mixture was heated to reflux, and became clear. After 4 h, the solvent was removed. A solution of 1-O-acetyl 3,5-di-O-benzoyl-2-deoxy-2-fluoro-D-arabinofuranose (30 mg, 0.072 mmol) in carbon tetrachloride (2 mL) was added followed by TMS-triflate (20 μL, 0.11 mmol, 1.5 eq). The flask which had contained the dry sugar was then rinsed with another aliquot (1.5 mL) of carbon tetrachloride, which was added. The reaction mixture was stirred at reflux for 20 h until TLC indicated no further change. The mixture was poured onto a short column of silica gel and eluted with 0.5% triethylamine in chloroform. The separation of the anomers was achieved by a subsequent longer column of neutralized silica using chloroform as eluent. The less-polar compound 17 was isolated as an amorphous solid (15.8 mg, 47%): ¹H NMR (500 MHz, CDCl₃) δ 8.78 (br s, 1H, imide-NH) 8.1-7.4 (m, 10H, 2 Bz), 6.77 (dd, 1H, J_{H1′-H2′}=4.0 Hz, J_{H1′-F2′}=23 Hz, H1′), 5.88 (ddd, 1H, J_{H2′-H3′}=2.5 Hz, J_{H3′-F2′}=9.6 Hz, J_{H3′-H4′}=2.0 Hz, H3′), 5.76 (d, 1H, J_{H5-H6}=8.2 Hz, H5), 5.27 (ddd, 1H, J_{H1′-H2′}=4.0 Hz, J_{H2′-H3′}=2.5 Hz, J_{H2′-F2′}=49.6 Hz, H2′), 4.67 (m, 2H, H5′, 5″), 3.99 (m, 1H, H4′). ¹³C NMR (125 MHz, CDCl₃): δ 166.25, 164.90, 162.74, 150.94 (4 CO), 142.28 (d, J_{C6-F2′}=4.7 Hz, C6), 134.37, 133.75, 130.27, 130.04, 129.53, 128.96, 128.82, 128.48 (2 OBz), 102.94 (C5), 94.66 (d, J_{C2′-F2′}=189.9 Hz, C2′), 153.59 (d, J_{C3′-F2′}=27.4 Hz, C3′), 64.68 (d, J_{C5′-F2′}=5.3 Hz, C5′), 61.83 (d, J_{C1′-F2′}=16.8 Hz, C1′), 50.96 (C4′). Two pairs of NOESY crosspeaks (H6-H3′, H6-H5′) provide strong evidence for top-face uracil and therefore the p nucleoside. FAB-HRMS: calcd. for C₂₃H₁₉N₂O₆SF+H⁺: 471.1026; found: 471.1027.

2′-Deoxy-2′-fluoro-4′-thio-β-D-arabinouridine (16, analogous to 13 but with uracil instead of thymine as a base moiety). To compound 17 (173 mg, 0.37 mmol) was added a 2M solution of ammonia in cold methanol (30 mL, 60 mmol). The reaction mixture was capped with a rubber septum and allowed to stir for 48 h. It was then evaporated to dryness, adsorbed onto silica and loaded onto a short column of neutralized silica gel. Dichloromethane containing 0-5% methanol was used to elute compound 16 as a solid (92 mg, 95%). ¹H NMR (400 or 500 MHz, methanol-d₄): δ 8.30 (dd, 1H, J_{H6-F2′}=1.6 Hz, J_{H6-H5}=8.4 Hz, H6), 6.41 (dd, 1H, J_{H1′-H2′}=5.6 Hz, J_{H1′-F2′}=11.6 Hz, H1′), 5.71 (d, 1H, J_{H6-H5}=8.4 Hz, H5), 5.00 (ddd, 1H, J_{H1′-H2′}=5.6 Hz, J_{H2′-F2′}=51.0 Hz, J_{H2′-H3′}=5.7 Hz, H2′), 4.36 (ddd, 1H, J_{H3′-H4′}=5.8 Hz, J_{H3′-F2′}=11.6 Hz, H3′), 3.82 (m, 2H, H5′, H5″), 3.33 (m, 1H, H4′). ¹³C NMR (125 MHz, methanol-d₄): δ 164.75, 151.55 (C2, C4), 143.31 (d, J_{F2′-C6}=2.3 Hz, C6), 101.07 (C5), 96.27 (d, J_{F2′-C2′}=193.8 Hz, C2′), 73.55 (d, J_{F2′-C3′}=23.6 Hz, C3′), 61.34 (d, J_{F2′-C5′}=2.4 Hz, C5′), 58.93 (d, J_{F2-C1′}=16.8 Hz, C1′), 51.88 (d, J_{F2′-C4′}=3.8 Hz, C4′). Two pairs of NOESY crosspeaks (H6-H3′, H6-H5′) provided strong evidence for top-face uracil and therefore the β nucleoside. FAB-HRMS: calcd. for C₉H₁₁N₂O₄SF+H⁺: 263.0502; found: 263.0501.

### Example 2

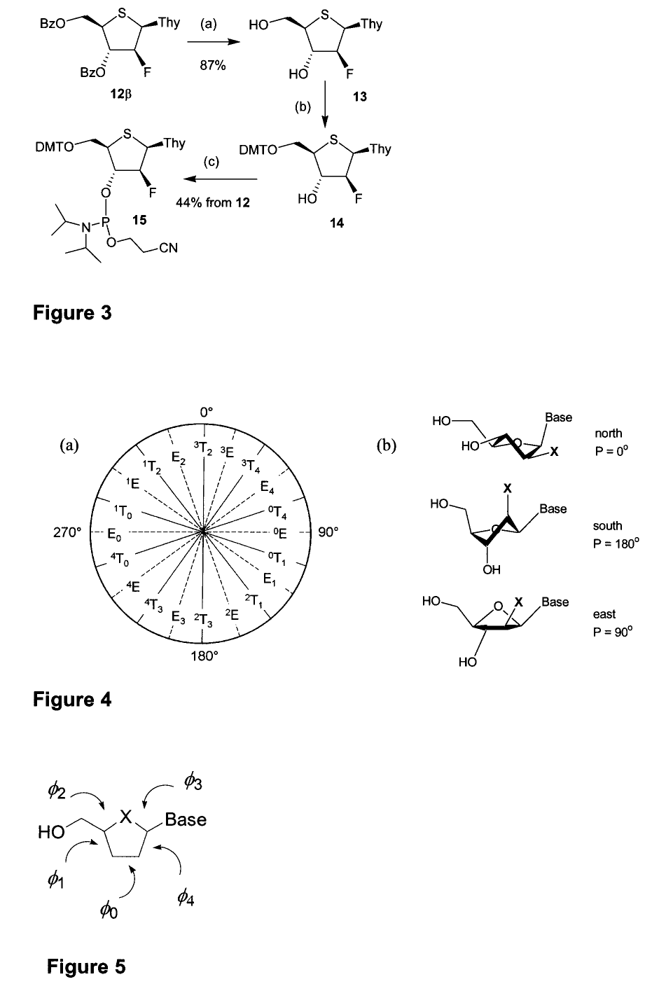

### Solid Phase Synthesis of Oligonucleotides (FIG. 3)

To prepare the nucleotide for use in solid phase synthesis, the 5′-hydroxyl group was protected using either 4-monomethoxytrityl (MMT) or 4,4′-dimethoxytrityl (DMT) chloride, but the latter required significantly shorter reaction times and was preferred. Phosphitylation of the tritylated compound 14 using bis(diisopropylamino)-β-cyanoethylphosphoramidite in the presence of diisopropylammonium tetrazolide, followed by precipitation from cold hexanes, gave the phosphoramidite 15 of suitable purity for solid phase oligonucleotide synthesis (FIG. 3). The tritylation and phosphitylation reactions were in general much slower for these modified nucleosides than for standard deoxyribo- or ribonucleosides.

Solid phase synthesis was carried out on a 1 μmol scale on an Applied Biosystems (ABI) 3400A synthesizer using the standard β-cyanoethylphosphoramidite chemistry according to published protocols (Wincott 2000) using 5-ethylthiotetrazole (0.25 M in acetonitrile) as activator. Phosphoramidites were prepared as 0.15 M solutions (RNA amidites) or 0.10 M solutions (DNA and 4′-thio amidites). Coupling times were extended to 10-30 minutes for modified nucleotides. Sequences were treated with 3:1 ammonium hydroxide:ethanol for 24 h at 55° C. to cleave from the solid support and deprotect. Sequences containing ribonucleotides were concentrated and further treated with Et₃N.3HF (100 μL) for 48 h at room temperature to remove 2′-O-silyl protecting groups. Sequences were then purified by anion exchange HPLC using 0-0.2 M LiClO₄ solution as eluent, followed by desalting on Sephadex G-25. Sequence purity was verified using 24% denaturing PAGE, loading 0.2 OD units of the oligomer.

Details of synthetic methods and characterization of tritylated compounds and phosphoramidites follow:

1-(2-Deoxy-2-fluoro-5-O-(4,4′-dimethoxytrityl)-4-thio-β-D-arabinofuranosyl)-thymine (14). 2′-Deoxy-2′-fluoro-4′-thio-β-D-arabinothymidine (13, 105 mg, 0.40 mmol) was coevaporated three times with pyridine. Dry pyridine (10 mL) was added, followed by 95% dimethoxytrityl chloride (198 mg, 0.56 mmol). Half of the solvent was removed, heating the flask slightly on a rotary evaporator. The reaction was allowed to stir for 44 h when TLC indicated virtual completion of the reaction. It was then diluted with dichloromethane (50 mL) and washed with saturated aqueous NaHCO₃ (2×50 mL); the aqueous layers were then washed with dichloromethane (2×50 mL). The organic layers were combined and concentrated. The residue was purified by preparative TLC (eluent 3.5% methanol, 0.2% triethylamine in dichloromethane) to yield 14 (260 mg, 106%). In spite of the impurities detected by the excess yield and by TLC, this product was a stable white foam and was used directly for the next step. ¹H NMR (400 MHz, acetone-d₆): δ 10.20 (br s, 1H, imide H-3), 7.60 (dd, 1H, J_{H6-F2′}=J_{H6-Me5}=1.4 Hz, H6), 7.60-6.90 (m, 14H, trityl), 6.52 (dd, 1H, J_{H1′-H2′}=5.2 Hz, J_{H1′-F2′}=15.2 Hz, H1′), 5.21 (br s, 1H, OH), 5.03 (ddd, 1H, J_{H2′-F2′}=50.8 Hz, J_{H2′-H3′}=5.2 Hz, H2′), 4.49 (ddd, 1H, J_{H3′-F2′}=11.5 Hz, J_{H3′-H4′}=4.8 Hz, H3′) 3.79 (s, 6H, 2 OCH₃), 3.62-3.43 (m, 3H, H4′, H5′, H5″), 1.74 (d, 3H, CH₃).

1-(3-O-(β-Cyanoethyl-N,N-diisopropylphosphoramidic)-2-deoxy-2-fluoro-5-O-(4,4′-dimethoxytrityl)-4-thio-β-D-arabinofuranosyl)-thymine (15). The crude compound 14 (260 mg) was coevaporated with dichloromethane and dried overnight over P₂O₅. It was then dissolved in dichloromethane (2 mL) and anhydrous diisopropylammonium tetrazolide (161 mg, 0.94 mmol) was added. Finally, 2-cyanoethyl-N,N,N′,N′-tetraisopropylphosphordiamidite (202 μL, 184 mg, 0.61 mmol) was added via syringe under a nitrogen atmosphere. The suspension was stirred for 68 h. A column was packed using neutralized silica in hexanes, and the reaction mixture was poured directly onto it. After elution in hexanes containing 10-50% ethyl acetate and 1% triethylamine, the fractions containing product were concentrated, and the product precipitated from cold hexanes to yield 15 as a white foam (151 mg, 44% over two steps). The mixture of two diastereomers at phosphorus led to complex ¹H and ¹³C NMR spectra. 31P NMR (81 MHz, acetone-d₆): δ 151.9 (d, J_{F-P}=6.2 Hz), 151.3 (d, J_{F-P}=3.4 Hz). FAB-HRMS: Calcd for C₄₀H₄₈N₄O₇FPS+K⁺: 817.2602; Found: 817.2606.

The uracil congener was prepared analogously, as follows:

2′-Deoxy-2′-fluoro-5′-O-(4-methoxytrityl)-4′-thio-β-D-arabinouridine (18; analogous to 14 but with uracil instead of thymine as a base moiety). 2′-Deoxy-2′-fluoro-4′-thio-β-D-arabinouridine (16, 105 mg, 0.40 mmol) was coevaporated three times with pyridine and left in a vacuum dessicator for 48 h. Monomethoxytrityl chloride (154 mg, 0.50 mmol, 1.25 eq.) was added along with a magnetic stir bar and septum, and the flask was flushed with nitrogen. Pyridine (4 mL) was then added via syringe and the reaction was allowed to stir. TLC showed that it had progressed to about 50% completion after 5 h and did not proceed further. Another aliquot of MMT-Cl (0.6 eq) was therefore added. After 72 h the reaction had stopped again; a few crystals of DMAP were added and the volume reduced by about half. The following day a third aliquot of MMT-Cl (0.5 eq) was added. The reaction reached completion after 7 days. Methanol (1 mL) and a small amount of neutralized silica were then added and the reaction mixture evaporated to dryness. The product was purified by preparative TLC (eluent 5% methanol, 0.1% triethylamine in dichloromethane) to yield compound 18 as a white foam (154 mg, 74%). ¹H NMR (500 MHz, acetone-d₆): δ10.3 (s, 1H, imide H-3), 7.90 (d, 1H, J_{H6-H5}=7.5 Hz, H6), 7.6-6.9 (m, 14H, MMT), 6.50 (dd, 1H, J_{H1′-H2′}=4.9 Hz, J_{H1′-F2′}=13.7 Hz, H1′), 5.51 (d, 1H, J_{H6-H5}=7.5 Hz, H5), 5.22 (br s, 1H, OH), 5.05 (ddd, 1H, J_{H1′-H2′}=4.9 Hz, J_{H2′-F2′}=51.0 Hz, J_{H2′-H3′}=5.0 Hz, H2′), 4.54 (m, 1H, H3′) 3.79 (s, 3H, OMe), 3.57-3.52 (m, 3H, H4′, H5′, H5″) ¹³C NMR (125 MHz, acetone-d₆): δ 162.84, 159.19, 151.16, 144.70, 144.62, 142.47, 135.26, 130.76, 128.68, 128.10, 127.30, 113.36, 101.72 (C5), 96.35 (d, J_{C2-F2′}=192.3 Hz, C2′), 87.07 (OCAr₃), 74.70 (d, J_{C3′-F2′}=23.7 Hz, C3′), 63.99 (d, J_{C5′-F2′}=2.3 Hz, C5′), 58.86 (d, J_{C1′-F2′}=16.0 Hz, C1′), 54.94 (OMe), 50.85 (d, J_{C4′-F2′}=3.8 Hz, C4′). FAB-HRMS: calcd. for C₂₉H₂₇N₂O₅SF+K⁺: 573.1262; found: 573.1261.

2′-Deoxy-2′-fluoro-3′-O-(β-cyanoethyl-N,N-diisopropylphosphoramidic)-5′-O-(4-methoxytrityl)-4′-thio-β-D-arabinouridine (19; analogous to 15 but with uracil instead of thymine as a base moiety). Compound 18 (155 mg, 0.29 mmol) was dried over P₂O₅ for several days, coevaporated with dry dichloromethane halfway through this period. It was then dissolved in dichloromethane (2 mL) and anhydrous diisopropylammonium tetrazolide (102 mg, 0.60 mmol, 2.0 eq) was added. Finally, 2-cyanoethyl-N,N,N′,N′-tetraisopropylphosphordiamidite (115 μL, 0.35 mmol) was added via syringe under a nitrogen atmosphere. The suspension was stirred for 46 h. The reaction mixture was loaded onto a column of triethylamine-neutralized silica and was purified by flash chromatography (using hexanes-ethyl acetate-triethylamine as eluent) to yield 19 as a foam (138 mg, 65%), collected as pure amidite diastereomers. Another fraction was isolated containing a mixture of starting material and product, and was phosphitylated again to yield a further 10 mg of product, for a total yield of 70%. For the faster-moving diastereomer: ³¹P NMR (81 MHz, acetone-d₆. δ 152.2 (d, J_{F-P}=6.5 Hz). ¹H NMR (500 MHz, acetone-d₆): δ10.19 (br s, 1H, H3 (uracil N3-H)), 7.86 (dd, 1H, J_{H6-H5}=8.0 Hz, J_{H6-F2′}=1.7 Hz, H6), 7.54-6.91 (m, 14H, trityl), 6.51 (dd, 1H, J_{H1′-H2′}=5.0 Hz, J_{H1′-F2′}=14.5 Hz, H1′), 5.49 (d, 1H, J_{H6-H5}=8.0 Hz, H5), 5.14 (ddd, J_{H1′-H2′}=5.0 Hz, J_{H2′-F2′}=50.5 Hz, J_{H2′-H3′}=4.6 Hz, H2′), 4.70 (m, 1H, H3′), 3.81 (s, 3H, OMe of MMT), 3.80-3.51 (m, 7H; H4′, H5′, H5″, OCH₂ of cyanoethyl, 2 NCH(CH₃)₂), 2.66 (t, 2H, J=6.2 Hz), 1.20, 1.191, 1.187, 1.17 (4 s, 12H, 2 NCH(CH₃)₂). ¹³C NMR (125.7 MHz, acetone-d₆): δ 162.53, 159.23, 151.04, 144.61, 144.53, 135.19 (C2, C4, 4 tertiary aromatic carbons of MMT), 142.19 (d, J_{C6-F2′}=2.9 Hz, C6), 130.80, 128.72, 128.71, 128.11, 127.35, (aromatic carbons of MMT), 118.77 (CN), 113.35 (aromatic carbon of MMT), 101.86 (CS), 95.56 (dd, J_{C2′-F2′}=193.8 Hz, J_{C2′-P}=3.6 Hz, C2′), 87.20 (OCAr₃), 76.48 (dd, J_{C3′-P}=16.2 Hz, J_{C3′-F2′}=24.3 Hz, C3′), 63.99 (d, J_{C5′-F2′}=3.6 Hz, C5′), 59.18, 59.03, 58.90, 58.76 (4 signals due to iPr methyls), 54.94 (OMe), 50.53 (dd, J_{C4′-F2′}≈J_{C4′-P}≈3 Hz, C4′), 43.40 (d, J_{C-P}=12.6 Hz, OCH₂CH₂CN), 24.27, 24.21, 24.16, 24.10 (4 Me of ⁱPr) ESI-MS: calcd for C₃₈H₄₄FN₄O₆PS+Na, 757.3; found, 757.0. Slower-moving diastereomer: ³¹P NMR (81 MHz, acetone-d₆): δ 151.4 (d, J_{F-P}=3.7 Hz) ¹H and ¹³C NMR very similar to those for the first diastereomer. Signals corresponding to the iPr and cyanoethyl groups were, predictably, those for which the largest differences were observed. ESI-MS: calcd for C₃₈H₄₄FN₄O₆PS+Na, 757.3; found, 757.1. NOESY spectra provided no useful information for identifying the stereochemistry of the two diastereomers.

### Example 3

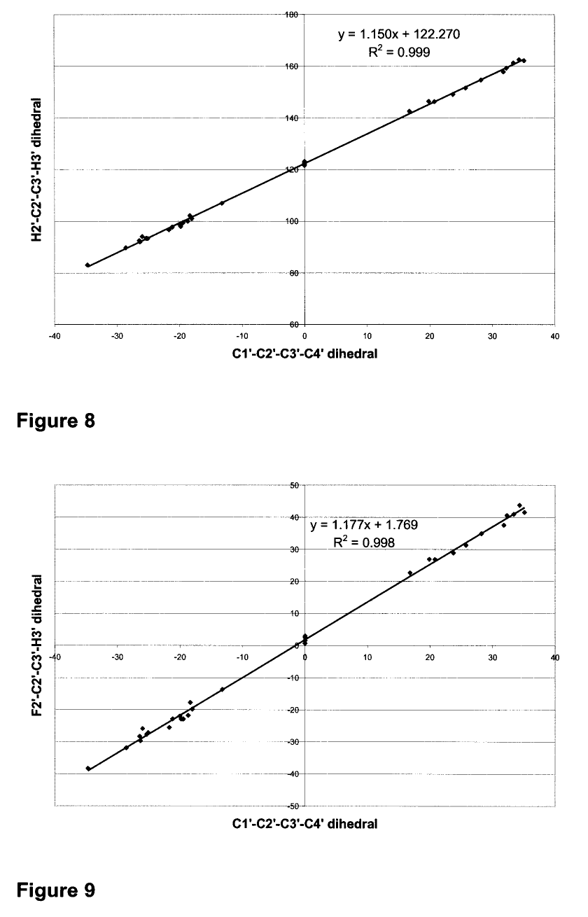

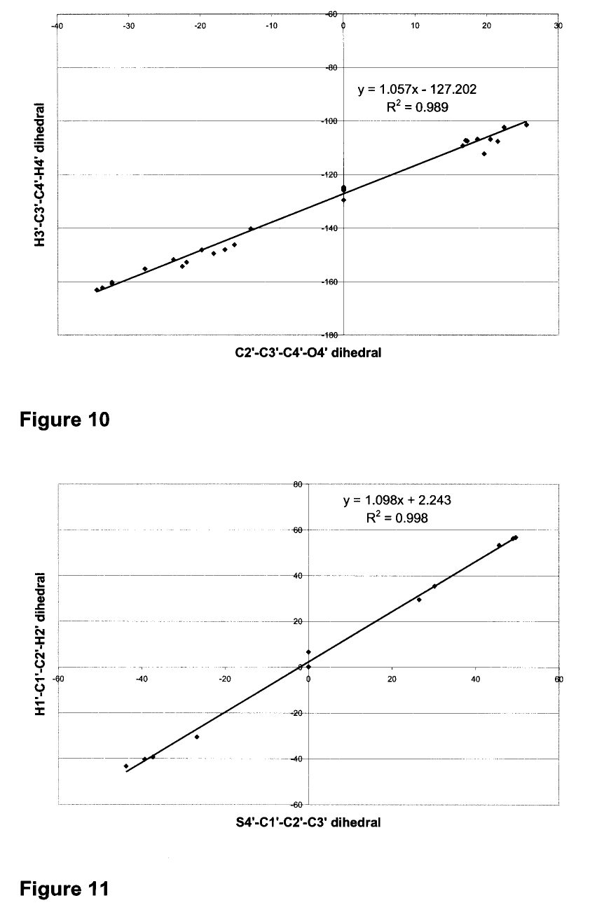

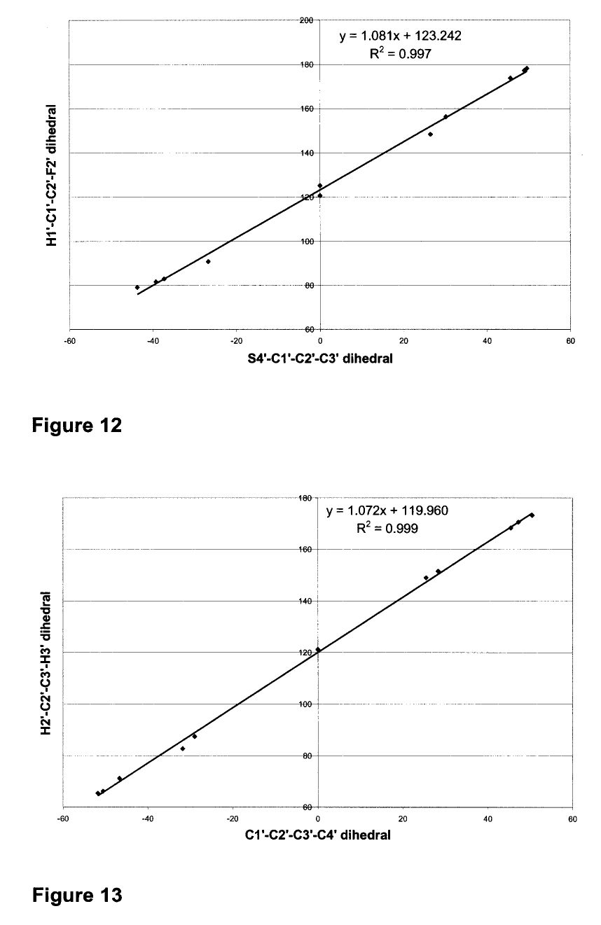

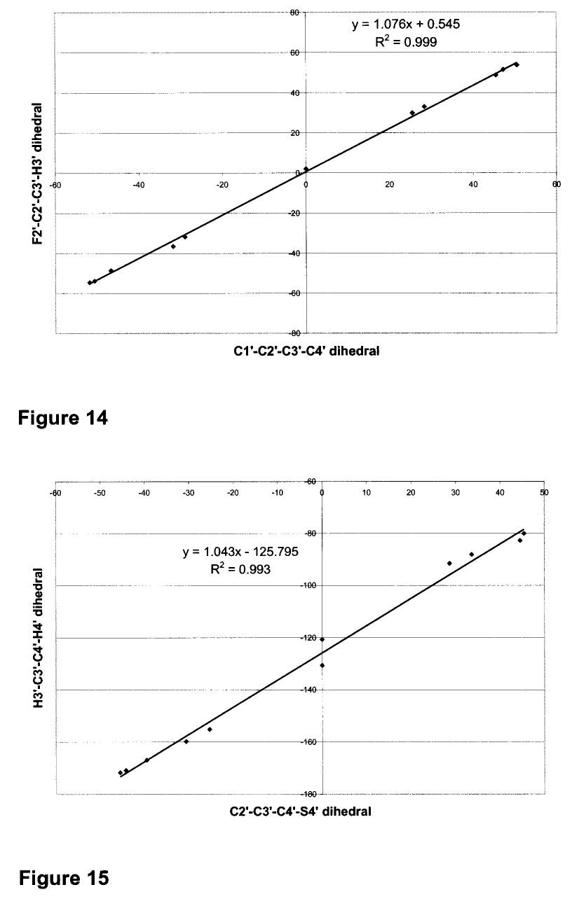

### Conformational Analysis of Nucleosides (FIGS. 4-15)

The conformational parameters of a nucleoside or other furanoside can be described using two parameters, namely the phase angle P and degree of maximum puckering φₘₐₓ (Altona et al. 1972) The value of P takes on an intuitive meaning when it is represented on a “pseudorotational wheel” as shown in FIG. 4.

The vicinal proton-proton and proton-fluorine coupling constants of the fully deprotected nucleoside 13 were examined and compared with those of its 4′-oxygen congener 16 (Table 2). According to the Karplus equation, northern conformers of arabino sugars have large values of ³J_{H2′-H3′} and ³J_{H3′-H4′}, while southern conformers have large values of ³J_{H1′-F2′}, since the nuclei are nearly antiperiplanar in all these cases. Taken together, the changes in these ³J values showed that a northern conformer was preponderant for the 4′-thionucleoside.

A large decrease of 7.5 Hz was observed in ³J_{F2′-H3′} upon changing the ring heteroatom from oxygen to sulfur. One possible explanation for the large ³J_{F2′-H3′} in the 4′-oxo species would be a contribution from an eastern conformer, in which F-2′ and H-3′ are eclipsed. This putative eastern conformation would be less significant for the 4′-thio species according to the reduced value of ³J_{F2′-H3′}.

We further used the PSEUROT 6.3 program (van Wijk et al, Leiden Institute of Chemistry, Leiden University, 1999), which is able to account for a two-state equilibrium and provide the pseudorotational parameters for two interconverting conformers. Detailed, empirically-derived data was not available for either nucleoside 13 or 16, and we therefore undertook a PSEUROT study of both nucleosides.

Several sets of parameters are necessary for the PSEUROT calculations. Valence angles are not perfectly tetrahedral, and an equation is needed to relate the external torsion angles (therefore the vicinal coupling constants) to the internal torsion angles (therefore the pseudorotational parameters P and φₘₐₓ). These two sets of angles are related as follows:

φⱼ^{exf}=Aⱼφⱼ+Bⱼ

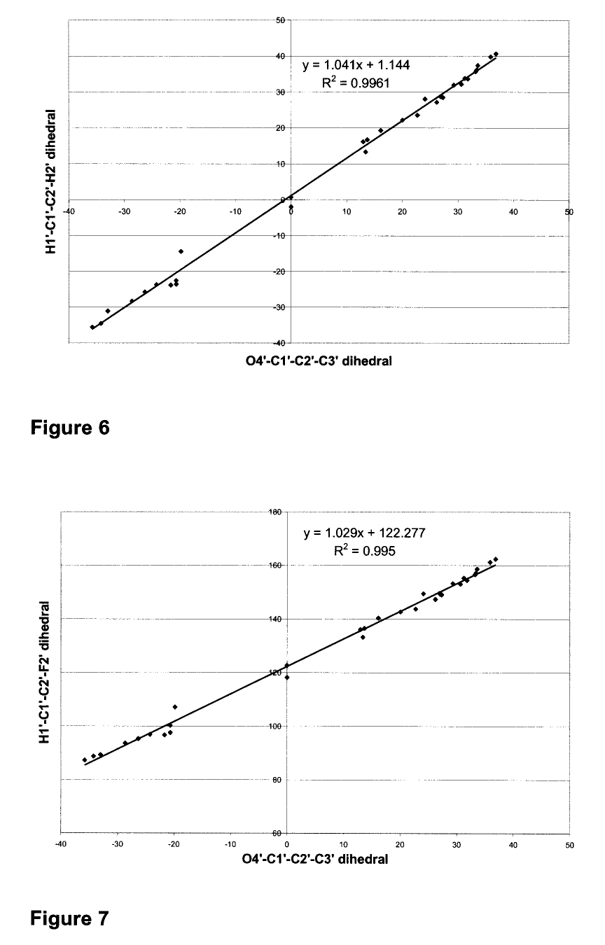

for j=0, . . . , 4. The definitions of the internal torsion angles are shown in FIG. 5. As these parameters were unknown for 2′-fluoroarabino or 2′-fluoro-4′-thioarabino configurations, we obtained them from Density Functional Theory (DFT) calculations (Table 3 and FIGS. 6-15).

A second set of parameters helps compensate for the non-equilateral nature of the rings. These parameters, αⱼ and εⱼ, named after Ernesto Diez, are used to modify the classical pseudorotation equations (Diez et al. 1984). Thus, in place of the standard pseudorotation equation,

φⱼ=φₘₐₓ cos(P+144°(j))

the equation is extended to yield,

φⱼ=αⱼφₘₐₓ cos(P+εⱼ+144°(j))

Including the αⱼ and εⱼ parameters in calculations on 4′-thionucleosides is particularly important because of their greater deviation from equilateral geometry. These parameters were therefore obtained for both systems studied by least squares minimization using the DFT-calculated structures mentioned above and the program FOURDIEZ (part of the PSEUROT suite of programs) (Table 4).

A generalized Karplus equation has been developed for ¹H-¹⁹F couplings, and proved to be useful for this work (Thibaudeau et al. 1998). However, since the ¹H-¹⁹F coupling constant is not as well characterized as the ¹H-¹H coupling constant, our initial PSEUROT calculations were carried out using only the three ¹H-¹H coupling values. To identify all possible solutions, 2400 consecutive calculations were carried out with different initial values of the five pseudorotational parameters, optimizing three of them at a time. The results were sorted by their rms error and the best several hundred solutions were examined carefully. Multiple possible solutions emerged.

The regions of pseudorotational space that gave low rms error (0.00 to 0.02 Hz for 4′S-FMAU, 0.00 to 0.50 Hz for FMAU) are shown in table 5. The 4′-thio compound 13 showed three distinct regions, all with very low rms error, but two of which included conformers in the western hemisphere that are highly unlikely according to DFT calculations and precedent. Its 4′-oxygen congener 14 showed one very broad region with higher rms error. The lowest rms error obtained within this general region was for a physically unlikely situation (φ_{maxII}=52°, which is too large for a 4′-oxygen furanose) but other more feasible sets of parameters were found in the same region.

To differentiate between these possible solutions and to refine the structures, the ¹H-¹⁹F coupling information was included. Each of the possible regions from the initial calculations was taken in turn as the starting point for the calculations. Inclusion of the fluorine couplings led to one set of pseudorotational parameters for the 4′-thionucleoside 13 being easily identified (Table 6). For the 4′-oxo nucleoside 16, the solution of best fit corresponded to a very unlikely arrangement, with the two conformers showing drastically different φₘₐₓ values and the second conformer too highly puckered for a 4′-oxo nucleoside. (The DFT calculations undertaken for the parametrization of PSEUROT confirmed that the replacement of O4′ by S causes the value of φₘₐₓ to increase by 10-15°.) Therefore, the calculations were also carried out constraining the φₘₐₓ of both conformers to 36°, a likely value according to the computed structures. The phase angles and mole fractions obtained from these two sets of calculations were similar; both results are listed in Table 6.

Whichever of the two solutions best describes nucleoside 14, it is clear that a northern pseudorotamer is preponderant for 13, while 16 is dominated by a conformer remarkably close to the southeast (see FIG. 4). It is of interest to note that whereas 4′S-FMAU (13) adopts predominantly the north conformation, the 2′-deoxynucleoside, i.e., 4′-thiothymidine (4′S-dT), adopts a south conformation in the solid state and a predominantly south conformation in solution (Koole et al. 1992).

### Key PSEUROT Input Files:

A: Initial “MANY” input file for FMAU.

2′F-ANA
CTRL MAXIT 25 TRIM 0.1 RCNV 0.5 MANY 6
DATA 3

| | | | | | | | |
|---|---|---|---|---|---|---|---|
| 1′-2′ | −144.0 | 1.041 | 1.144 | 0.56 | 0.70 | 0.62 | 1.37 |
| 2′-3′ | 0.0 | 1.150 | 122.27 | 1.37 | 0.62 | 1.25 | 0.62 |
| 3′-4′ | 144.0 | 1.0565 | −127.2 | 0.62 | 1.25 | 0.70 | 0.68 |

DIEZ

| | |
|---|---|
| 0.995 | −1.409 |
| 0.981 | −0.229 |
| 0.988 | 1.621 |

TSET 1

| | | | | | |
|---|---|---|---|---|---|
| 25 C | 4.0 | 2.85 | 5.0 | | |
| START | 18.0 | 36.0 | 162.0 | 36.0 | .50 |
| FITF | 10101 | | | | |

B: Final input files (after refining FCC and HCC angles and starting parameters based on the output from the “MANY” calculations) for FMAU.

2′F-ANA - with refined HCC, FCC - no Diez - F weighting 0.2 - pucker
36, fitf 10101
CTRL MAXIT 5000 TRIM 0.1 RCNV 0.5 PRINT 1
DATA 5

| | | | | | | | |
|---|---|---|---|---|---|---|---|
| 1′-2′ | −144.0 | 1.041 | 1.144 | 0.56 | 0.70 | 0.62 | 1.37 |
| 2′-3′ | 0.0 | 1.150 | 122.270 | 1.37 | 0.62 | 1.25 | 0.62 |
| 3′-4′ | 144.0 | 1.057 | −127.202 | 0.62 | 1.25 | 0.70 | 0.68 |
| 1′-F | −144.0 | 1.029 | 122.277 | 0.56 | 0.70 | 0.00 | 0.62 |
| F-3′ | 0.0 | 1.177 | 1.769 | 0.62 | 0.00 | 1.25 | 0.62 |

HETERO

| | | | | | | |
|---|---|---|---|---|---|---|
| 0 | 110 | 110 | 0 | 1.0 | | |
| 0 | 110 | 110 | 0 | 1.0 | | |
| 0 | 110 | 110 | 0 | 1.0 | | |
| 1 | 114.4 | 111.8 | −3.72 | 0.2 | | |
| 1 | 109.6 | 109.0 | −3.72 | 0.2 | | |
| cagp1 | 40.61 | −4.22 | 5.88 | −1.27 | −6.20 | 0.20 |

TSET 1

| | | | | | |
|---|---|---|---|---|---|
| 25 C | 4.0 | 2.85 | 5.0 | 16.85 | 19.56 |
| START | −7.6 | 36.0 | 124.2 | 36.0 | .69 |
| FITF | 10101 | | | | |

2′F-ANA - with refined HCC, FCC - no Diez - F weighting 0.2 - all
fitflags free
CTRL MAXIT 5000 TRIM 0.1 RCNV 0.5 PRINT 1
DATA 5

| | | | | | | | |
|---|---|---|---|---|---|---|---|
| 1′-2′ | −144.0 | 1.041 | 1.144 | 0.56 | 0.70 | 0.62 | 1.37 |
| 2′-3′ | 0.0 | 1.150 | 122.270 | 1.37 | 0.62 | 1.25 | 0.62 |
| 3′-4′ | 144.0 | 1.057 | −127.202 | 0.62 | 1.25 | 0.70 | 0.68 |
| 1′-F | −144.0 | 1.029 | 122.277 | 0.56 | 0.70 | 0.00 | 0.62 |
| F-3′ | 0.0 | 1.177 | 1.769 | 0.62 | 0.00 | 1.25 | 0.62 |

HETERO

| | | | | | | |
|---|---|---|---|---|---|---|
| 0 | 110 | 110 | 0 | 1.0 | | |
| 0 | 110 | 110 | 0 | 1.0 | | |
| 0 | 110 | 110 | 0 | 1.0 | | |
| 1 | 114.4 | 111.8 | −3.72 | 0.2 | | |
| 1 | 109.6 | 109.0 | −3.72 | 0.2 | | |
| cagp1 | 40.61 | −4.22 | 5.88 | −1.27 | −6.20 | 0.20 |

TSET 1

| | | | | | |
|---|---|---|---|---|---|
| 25 C | 4.0 | 2.85 | 5.0 | 16.85 | 19.56 |
| START | −7.6 | 38.0 | 124.2 | 38.0 | .69 |
| FITF | 11111 | | | | |

C: Initial “MANY” input file for 4′S-FMAU.

2′F-4′S-ANA
CTRL MAXIT 1000 TRIM 0.1 RCNV 0.5 MANY 6
DATA 3

| | | | | | | | |
|---|---|---|---|---|---|---|---|
| 1′-2′ | −144.0 | 1.098 | 2.019 | 0.56 | 0.70 | 0.62 | 1.37 |
| 2′-3′ | 0.0 | 1.068 | 120.013 | 1.37 | 0.62 | 1.25 | 0.62 |
| 3′-4′ | 144.0 | 1.064 | −125.397 | 0.62 | 1.25 | 0.70 | 0.68 |

DIEZ

| | |
|---|---|
| 1.0325 | 3.495 |
| 1.0363 | −0.0745 |
| 1.0284 | −3.575 |

TSET 1

| | | | | | |
|---|---|---|---|---|---|
| 25 C | 6.0 | 7.1 | 7.0 | | |
| START | 18.0 | 45.0 | 162.0 | 45.0 | .5 |
| FITF | 10101 | | | | |

D: Final input file (after refining FCC and HCC angles) for 4′S-FMAU.

2′F-4′S-ANA - final
CTRL MAXIT 5000 TRIM 0.1 RCNV 0.5 PRINT 1
DATA 5

| | | | | | | | |
|---|---|---|---|---|---|---|---|
| 1′-2′ | −144.0 | 1.098 | 2.243 | 0.56 | 0.70 | 0.62 | 1.37 |
| 1′-F | −144.0 | 1.081 | 123.242 | 0.56 | 0.70 | 0.00 | 0.62 |
| 2′-3′ | 0.0 | 1.072 | 119.960 | 1.37 | 0.62 | 1.25 | 0.62 |
| F-3′ | 0.0 | 1.076 | 0.545 | 0.62 | 0.00 | 1.25 | 0.62 |
| 3′-4′ | 144.0 | 1.043 | −125.795 | 0.62 | 1.25 | 0.70 | 0.68 |

DIEZ

| | |
|---|---|
| 1.0323 | 3.478 |
| 1.0323 | 3.478 |
| 1.0354 | −0.0573 |
| 1.0354 | −0.0573 |
| 1.0298 | −3.615 |

HETERO

| | | | | | | |
|---|---|---|---|---|---|---|
| 0 | 110 | 110 | 0 | 1.0 | | |
| 1 | 113.2 | 110 | −3.72 | 0.2 | | |
| 0 | 110 | 110 | 0 | 1.0 | | |
| 1 | 109.8 | 110 | −3.72 | 0.2 | | |
| 0 | 110 | 110 | 0 | 1.0 | | |
| cagp1 | 40.61 | −4.22 | 5.88 | −1.27 | −6.20 | 0.20 |

TSET 1

| | | | | | |
|---|---|---|---|---|---|
| 25 C | 6.0 | 7.9 | 7.1 | 12.1 | 7.0 |
| START | −90.0 | 48.0 | 0.0 | 48.0 | .70 |
| FITF | 11111 | | | | |

### Example 4

### UV Thermal Denaturation Studies

UV thermal denaturation data were obtained on a Varian CARY 300 spectrophotometer equipped with a Peltier temperature controller. Equimolar amounts of complementary sequences (about 0.4 ODU of each strand) were combined, dried and rediluted in 1 mL of pH 7.2 buffer containing 140 mM KCl, 1 mM MgCl₂ and 5 mM NaHPO₄. Strands were annealed in the buffer at 95° C. for 5 minutes, slowly cooled down to 4° C. (over about 5 hours) then kept at 4° C. for several hours before measurements. Changes in absorbance at 260 nm were monitored upon heating. Melting temperatures were determined as the maxima of the first derivatives and are given in Tables 7-9.

It is noteworthy that 2′F-4′S-ANA tends to have reduced affinity for RNA. This relatively low affinity could be useful in siRNA applications, because of the importance of strand bias in the loading of RISC (Hohjoh 2004).

### Example 5

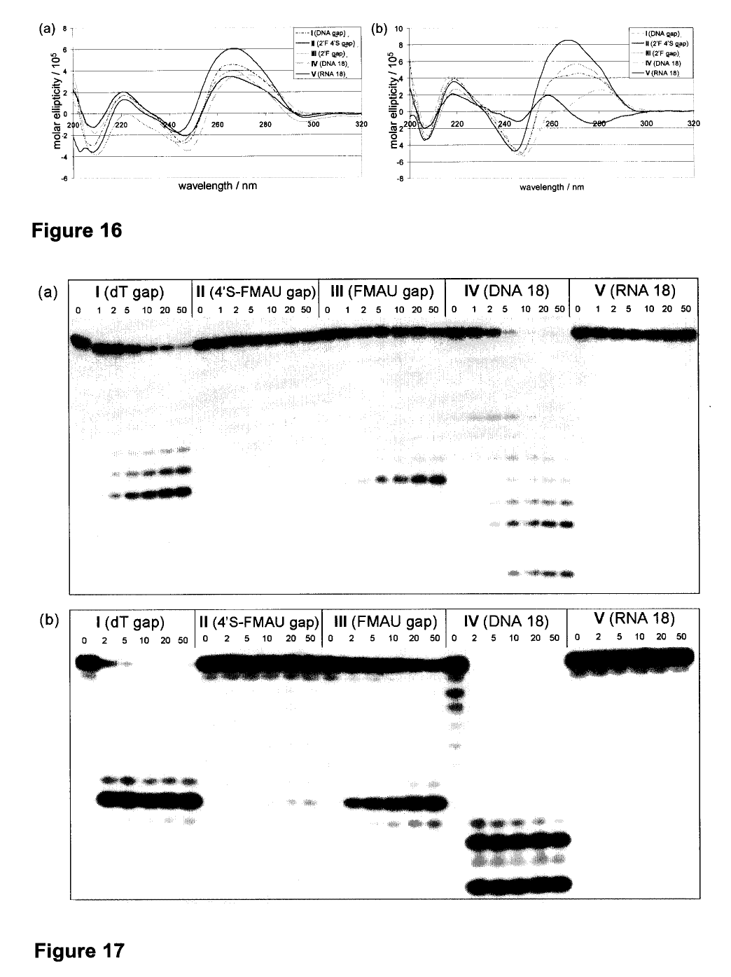

### Circular Dichroism Studies of Oligonucleotides (FIG. 16)

Hybrids comprising any one of sequences I-V bound to either ssRNA or ssDNA targets were further evaluated for possible variations in duplex structure via CD spectroscopy, in the region from 320-200 nm (FIG. 16). The spectra of all AON:RNA hybrids exhibit the characteristic A-form pattern, with the largest changes evident in the magnitude and positions of the positive Cotton effect at ca. 265 nm. The highest Cotton effect (molar ellipticity) observed corresponds to that of the pure RNA:RNA duplex (V:RNA). The Cotton effects of the 2′F-4′S-ANA gapmer (II):RNA duplex are blue-shifted, but the overall CD trace similarly indicates an A-form global geometry. The spectra of the AON:DNA hybrids, however, are much more varied in comparison. Most striking is the CD signature of the II:DNA duplex, which bears no similarity to either A- or B-form reference spectra. Of note, for example, are the negative peak at 280 nm, the cross-over at 270 nm, and the positive peak at 257 nm, all of which are unique to the II:DNA spectrum. The helical structure of this hybrid is apparently quite different from either A-form or B-form helices, thus supporting the notion that the increased S—C bond length, the smaller C—S—C bond angle or the more puckered ring causes a divergence from the classical helix structure, or might perturb the N-glycosidic orientation around the nucleotide sugars, thereby destacking the helix. The fact that greater structural distortions are observed with ssDNA instead of ssRNA targets (as measured by CD) may further point to this phenomenon, and is also likely to be related to the inherently greater flexibility of DNA over RNA targets. It is also probable that the greater structural distortion for the ssDNA target is related to the fact that the preferred conformation of the 4′S-FMAU nucleoside is in the north, thus more compatible with an RNA-like (A-form) structure.

### Example 6

### Ribonuclease H Activity Assays (FIG. 17)

The RNase H family comprises a class of enzymes that have the common property of recognizing and cleaving the RNA strand of AON:RNA hybrids having a conformation that is intermediate between the pure A- or B-form conformations adopted by dsRNA and dsDNA, respectively. Sugar geometries that fall within the eastern (O4′-endo) range within the AON have been postulated to actively induce RNase H-assisted RNA strand cleavage (Trempe et al. 2001). Chemical changes of the sugar constituents or alterations in the sugar conformation (e.g., orientation of the sugar to the base) or flexibility (e.g., DNA versus the more rigid 2′F-ANA analog) can all dramatically affect RNase H activation (Mangos et al. 2003).

It has been shown that an 18-mer chimera containing six central 2′-deoxyribonucleotides or 2′F-ANA nucleotides surrounded by native RNA wings is a substrate for RNase H (Lok et al. 2002). The RNA wings serve to ensure tight binding, and the central section is adequate to elicit RNase H activity. In this way, new modifications can be tested for a true effect on RNase H activity without compromising the binding properties of the oligonucleotide. Oligonucleotides I-V (Table 7) were assessed for their ability to elicit E. coli RNase HI and human RNase HII activity. As shown in FIG. 17, the control DNA oligomer IV and DNA gap I both promoted essentially complete degradation of the 5′-³²P-labeled RNA. As expected, the RNA duplex was not a substrate of RNase H. With the 2′F-ANA gap (III) the enzyme activity was somewhat lower compared to DNA, although significant cleavage (>50%) occurred after 50 min under these conditions, as previously observed (Lok et al. 2002). Negligible or no cleavage was observed for the 2′F-4′S-ANA modified (II):RNA hybrid. The ability of the various gaps to elicit E. coli RNase HI activity followed the order: DNA>2′F-ANA>>2′F-4′S-ANA≈RNA (FIG. 17A). The same trend was observed with the human enzyme (FIG. 17B). The lack of RNase H activity supported by 2′F-4′S-ANA is consistent with the northern conformation (C3′-endo) of this modification shown herein.

Experimental details for these assays:

The activity of E. coli RNase HI (USB Corporation, Cleveland, Ohio) was tested with antisense oligonucleotides under conditions recommended by the manufacturer (50 mM Tris-HCl, pH 7.5, 50 mM KCl, 25 mM MgCl₂, 0.25 mM EDTA, 0.25 mM DTT). The antisense and 5′-³²P labeled sense strands (Table 7) were combined in a 2:1 ratio and annealed by heating to 90° C. followed by slow cooling to room temperature. 2.5 Units (17 μg) of enzyme were incubated at 37° C. in the described buffer for 10 minutes, and 100 μl final volume reactions were initiated by addition of duplexed antisense/sense substrate to a concentration of 50 nM. Aliquots were removed at various times as indicated in FIG. 17 and quenched by the addition of an equal volume of loading buffer (98% deionized formamide, 10 mM EDTA, 1 mg/mL bromophenol blue, and 1 mg/mL xylene cyanol), followed by heating to 95° C. for 5 min. Cleavage products were resolved on 16% denaturing PAGE and visualized by autoradiography.

Human RNase HII was expressed and purified using a slight modification of the published procedure (Wu et al. 1999). The assays were performed analogously to that described above, using a 3:1 antisense:sense strand ratio, a buffer containing 60 mM Tris-HCl, pH 7.8, 60 mM KCl, 2.5 mM MgCl₂ and 2 mM DTT, and enzyme concentrations of 37 and 110 nM.

### Example 7

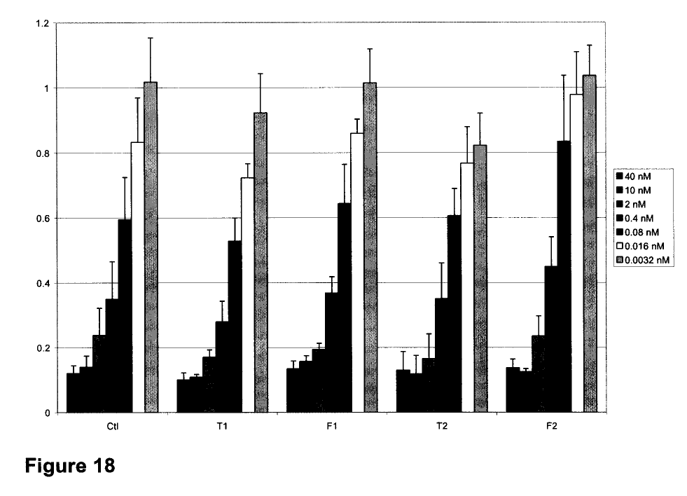

### RNA Interference Assays (FIGS. 18-20)

2′-fluoro-4′-thioarabinouridine was introduced at various positions into both strands of an siRNA sequence targeting the firefly luciferase gene (Tables 8-9). siRNAs containing FMAU at the same positions were used as controls, along with native RNA. The resulting modified duplexes were transfected into HeLa cells stably expressing firefly luciferase as follows:

HeLa X1/5 cells, expressing the firefly luciferase gene, were maintained and grown in EMEM supplemented with 10% fetal bovine serum (FBS), 2 mM L-glutamine, 1% non-essential amino acids, 1% MEM vitamins, 500 μl/ml G418, 300 μg/ml Hygromycin as described previously (Lok et al, 2002.). The day prior to transfection, 0.5×10⁵ cells were plated in each well of a 24-well plate. The next day, the cells were incubated with increasing amounts of siRNAs premixed with lipofectamine-plus reagent (Invitrogen) using 1 μL of lipofectamine and 4 μL of the plus reagent per 20 μmol of siRNA (for the highest concentration tested). For the siRNA titrations, each siRNA was diluted into dilution buffer (30 mM HEPES-KOH, pH 7.4, 100 mM KOAc, 2 mM MgOAc₂) and the amount of lipofectamine-plus reagent used relative to the siRNAs remained constant. 24 hours after transfection, the cells were lysed in hypotonic lysis buffer (15 mM K₃PO₄, 1 mM EDTA, 1% Triton, 2 mM NaF, 1 mg/ml BSA, 1 mM DTT, 100 mM NaCl, 4 μg/mL aprotinin, 2 μg/mL leupeptin and 2 μg/mL pepstatin) and the firefly light units were determined using a Fluostar Optima 96-well plate bioluminescence reader (BMG Labtech) using firefly substrate as described previously (Novac et al., 2004). The luciferase counts were normalized to the protein concentration of the cell lysate as determined by the DC protein assay (BioRad). Error bars represent the standard deviation of at least four transfections. Cotransfecting the siRNAs and the plasmid pCI-hRL-con expressing the Renilla luciferase mRNA (Pillai et al., 2005) in the same cell line showed no difference in expression of this reporter, demonstrating the specificity of the RNAi effects (data not shown). Results are summarized in Tables 8 and 9, and FIGS. 18-20.

The 2′F-4′S-ANA modification is generally well-tolerated by the RNAi machinery. The potencies of the 2′F-4′S-ANA and 2′F-ANA modified strands are comparable.

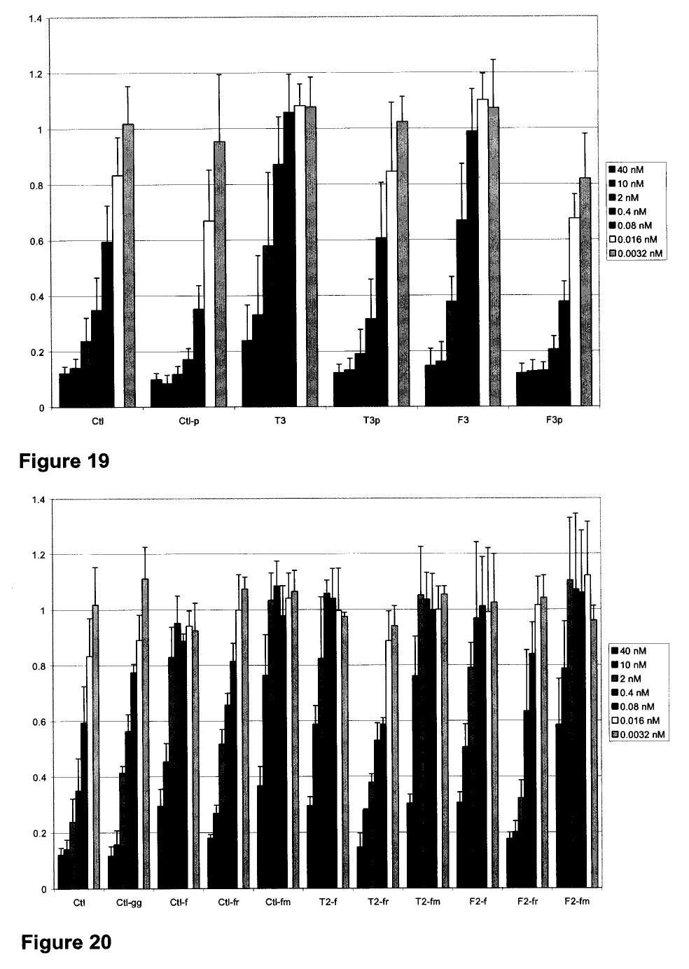

When the terminal pair of nucleotides of the antisense strand is modified by either one of the nucleotides under investigation in this study, the activity is significantly reduced. Chemical or enzymatic phosphorylation prior to transfection dramatically increased the activity of terminally-modified strands (FIG. 19). Even the control strand showed an improvement in potency upon 5′-phosphorylation.

It is significant that two 2′F-4′S-ANA and FANA modifications can be introduced at the 5′-terminus of the antisense strand, resulting in a strand with potency comparable to that of native RNA (duplexes T3p and F3p, Table 8).

The 2′F-4′S-ANA antisense modifications were tested in combination with various heavily-modified sense strands. We included a duplex with an all-2′F-ANA sense strand in our assays (duplexes Ctl-f, T2-f and F2-f, Table 9). To improve upon the activity of this strand, however, we made two other modifications: (1) a fully-modified 2′F-ANA sense strand containing two appropriately-placed mismatches (duplexes Ctl-fm, T2-fm and F2-fm), and (2) a sense strand was made containing 5 RNA inserts at its 3′-end (duplexes Ctl-fr, T2-fr and F2-fr). The 2-nucleotide 3′-overhang was left as 2′F-ANA to help provide 3′-exonuclease resistance. Results are given in FIG. 20.

In all cases, the “fr” type sense strand was the best heavily-modified sense strand, reaching levels of potency close to that of the control. It is interesting to note the synergy between 2′F-4′S-ANA and 2′F-ANA in the T2-fr duplex, which gave particularly good results.

TABLE 1
Anomeric ratio of nucleoside products for glycosylations with the
β-acetate 9β as starting material ( [symbol] ≦ 10%).

| | Dielectric constant | |
|---|---|---|
| | [CRC Handbook of | |
| | Chemistry and Physics, | |
| Solvent | 77^{th} ed.] | Product α:β ratio |
| CH₃CN | 37.5 | 3:1 |
| CH₂Cl₂ | 9.1 | 1.7:1 |
| CHCl₃ | 4.8 | 0.9:1 |
| CCl₄ | 2.2 | 0.7:1 |

TABLE 2
Vicinal ¹H-¹H and ¹H-¹⁹F coupling constants^{a} in 4′S-FMAU (13)
and FMAU (16) nucleosides in D₂O.

| | 4′S-FMAU (13) | FMAU (16) |
|---|---|---|
| H1′-H2′ | 6.0 | 4.0 |
| H1′-F2′ | 7.9 | 16.9 |
| H2′-H3′ | 7.1 | 2.9 |
| F2′-H3′ | 12.1 | 19.6 |
| H3′-H4′ | 7.0 | 5.0 |

^{a}In Hz.

TABLE 3
Aⱼ and Bⱼ parameters for 13 and 16.

| | |
|---|---|
| 4′S-FMAU (13) | FMAU (16) |

| | Aⱼ | Bⱼ^{a} | Aⱼ | Bⱼ^{a} |
|---|---|---|---|---|
| H_{1′}-H_{2′} | 1.098 | 2.24 | 1.041 | 1.14 |
| H_{1′}-F_{2′} | 1.081 | 123.24 | 1.029 | 122.28 |
| H_{2′}-H_{3′} | 1.072 | 119.96 | 1.150 | 122.27 |
| F_{2′}-H_{3′} | 1.076 | 0.54 | 1.177 | 1.77 |
| H_{3′}-H_{4′} | 1.043 | −125.80 | 1.057 | −127.20 |

^{a}In degrees.

TABLE 4
Diez parameters αⱼ and εⱼ for 13 and 16.

| | |
|---|---|
| 4′S-FMAU (13) | FMAU (16) |

| | αⱼ | εⱼ^{a} | αⱼ | εⱼ^{a} |
|---|---|---|---|---|
| φ₁ | 1.030 | −3.615 | 0.998 | 1.621 |
| φ₂ | 0.955 | −0.355 | 1.012 | 0.252 |
| φ₃ | 0.952 | 0.435 | 1.016 | −0.223 |
| φ₄ | 1.032 | 3.478 | 0.995 | −1.415 |
| φ₀ | 1.035 | −0.057 | 0.981 | −0.229 |

^{a}In degrees.

TABLE 5
General regions corresponding to mathematically
possible solutions of the initial PSEUROT calculations
(¹H-¹H coupling constants only.)

| | | | |
|---|---|---|---|
| Nucleoside | P_{I} (φ_{maxI})^{a} | P_{II} (φ_{maxII})^{a} | Ratio |

| | | | | | |
|---|---|---|---|---|---|
| 13 | −6 | (44) | 200 | (44) | 77:23 |
| 13 | −40 | (51) | 45 | (51) | 70:30 |
| 13 | −90 | (48) | 0 | (48) | 25:75 |
| 14 | −20 to 20 | (38) | 124 | (42-52) | 30:70 |

^{a}In degrees.

TABLE 6
Final results from PSEUROT calculations (including ¹H-¹⁹F
coupling constants) for 4′S-FMAU (13) and FMAU (16).

| | | | | RMS error |
|---|---|---|---|---|
| Nucleoside | P_{I} (φ_{maxI})^{a} | P_{II} (φ_{maxII})^{a} | Ratio | of the fit |
| 13 | −4 (44) | 199 (43) | 77:23 | 0.000 Hz |
| 16^{b} | −6 (36) | 126 (36) | 31:69 | 0.595 Hz |
| 16^{c} | −35 (39) | 116 (53) | 37:63 | 0.000 Hz |

^{a}In degrees.
^{b}With φₘₐₓ of both conformers constrained at 36°.
^{c}With no constraints on the minimization.

TABLE 7

UV thermal denaturation studies of modified
oligonucleotides (sequences also used for
circular dichroism and RNase H studies).^{a}

| | | |
|---|---|---|
| | Tₘ | Tₘ |
| | (RNA | (DNA |
| Sequence | target) | target) |

| | | | | |
|---|---|---|---|---|
| I | 5′-UGA CAU ttt ttt UCA CGU-3′ | (SEQ ID NO:2) | 60.0 | 51.0 |
| II | 5′-UGA CAU TTT TTT UCA CGU-3′ | (SEQ ID NO:3) | 51.0 | 36.0 |
| III | 5′-UGA CAU [symbol] [symbol] UCA CGU-3′ | (SEQ ID NO:4) | 62.0 | 50.1 |
| IV | 5′-tga cat ttt ttt tca cgt-3′ | (SEQ ID NO:5) | 42.1 | 55.5 |
| V | 5′-UGA CAU UUU UUU UCA CGU-3′ | (SEQ ID NO:6) | 59.1 | 40.2 |

^{a}Legend: RNA, dna, 2′F4′S-ANA, [symbol]
Complementary strands were as follows: RNA, 5′-ACG UGA AAA AAA AUG UCA-3′ (SEQ ID NO:1), DNA, 5′-acg tga aaa aaa atg tca-3′ (SEQ ID NO:7)

TABLE 8
siRNA sequences and thermal denaturation studies.^{a}

| | | |
|---|---|---|
| | Tₘ | IC₅₀ |
| Duplex | (° C.) | (nM) |

| | | | | |
|---|---|---|---|---|
| Ctl | 5′-GCUUGAAGUCUUUAAUUAAtt-3′ | (SEQ ID NO:8) | 62.3 | 0.10 |
| | 3′-ggCGAACUUCAGAAAUUAAUU-5′ | (SEQ ID NO:9) | | |
| Ctl-p | 5′-GCUUGAAGUCUUUAAUUAAtt-3′ | (SEQ ID NO:8) | n.d. | 0.04 |
| | 3′-ggCGAACUUCAGAAAUUAAUUp-5′ | (SEQ ID NO:10) | | |
| T1 | 5′-GCUUGAAGUCUUUAAUUAAtt-3′ | (SEQ ID NO:11) | 60.2 | 0.10 |
| | 3′-ggCGAACUUCAGAAAUUAAUU-5′ | (SEQ ID NO:9) | | |
| F1 | 5′-GCUUGAAGUCUUUAA [symbol] AAtt-3′ | (SEQ ID NO:12) | 63.0 | 0.20 |
| | 3′-ggCGAACUUCAGAAAUUAAUU-5′ | (SEQ ID NO:9) | | |
| T2 | 5′-GCUUGAAGUCUUUAAUUAAtt-3′ | (SEQ ID NO:8) | 57.2 | 0.25 |
| | 3′-ggCGAACUUCAGAAAUUAAUU-5′ | (SEQ ID NO:13) | | |
| F2 | 5′-GCUUGAAGUCUUUAAUUAAtt-3′ | (SEQ ID NO:8) | 60.0 | 0.73 |
| | 3′-ggCGAACU [symbol] CAGAAAUUAAUU-5′ | (SEQ ID NO:14) | | |
| T3 | 5′-GCUUGAAGUCUUUAAUUAAtt-3′ | (SEQ ID NO:8) | 62.0 | 1.4 |
| | 3′-ggCGAACUUCAGAAAUUAAUU-5′ | (SEQ ID NO:15) | | |
| T3p | 5′-GCUUGAAGUCUUUAAUUAAtt-3′ | (SEQ ID NO:8) | n.d. | 0.07 |
| | 3′-ggCGAACUUCAGAAAUUAAUUp-5′ | (SEQ ID NO:16) | | |
| F3 | 5′-GCUUGAAGUCUUUAAUUAAtt-3′ | (SEQ ID NO:8) | 62.1 | 1.3 |
| | 3′-ggCGAACUUCAGAAAUUAA [symbol] -5′ | (SEQ ID NO:17) | | |
| F3p | 5′-GCUUGAAGUCUUUAAUUAAtt-3′ | (SEQ ID NO:8) | n.d. | 0.05 |
| | 3′-ggCGAACUUCAGAAAUUAA [symbol] p-5′ | (SEQ ID NO:18) | | |

^{a}Legend: RNA, dna, 2′F-4′S-ANA, [symbol]
Sense strands are listed on top and antisense strands below.
Duplexes with names ending in “p” were 5′phosphorylated on the antisense strand (see text for details)

TABLE 9
Effect of significantly-modified sense strands with FAU
point modifications in the antisense strand.^{a}

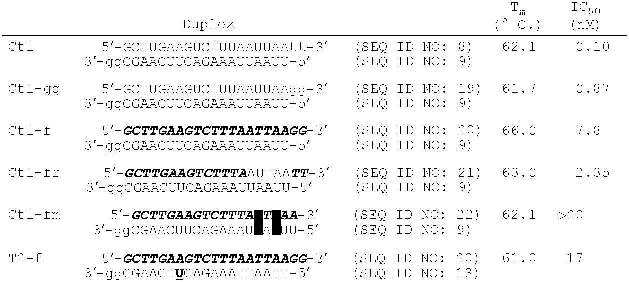

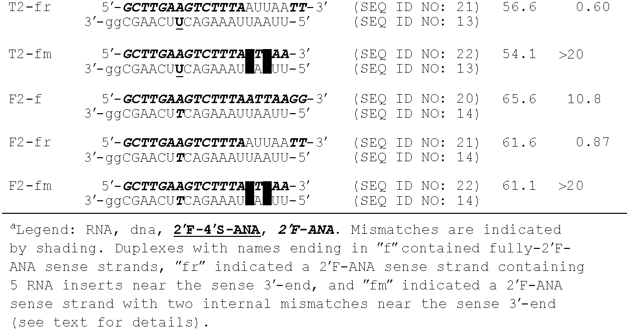

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

Throughout this application, various references are referred to describe more fully the state of the art to which this invention pertains. The disclosures of these references are hereby incorporated by reference into the present disclosure.

### REFERENCES

Altona, C. and M. Sundaralingam (1972). J. Am. Chem. Soc. 94(23): 8205-8212.
Diez, E., A. L. Esteban, et al. (1984). J. Mol. Struct. 125(1-2): 49-65.
Elbashir, S. M., W. Lendeckel, et al. (2001). Genes Dev. 15(2): 188-200.
Fire, A., S. Xu, et al. (1998). Nature 391(6669): 806-811.
Hohjoh, H. (2004). FEBS Lett. 557(1-3): 193-198.
Koole, L. H., J. Plavec, et al. (1992). J. Am. Chem. Soc. 114(25): 9936-9943.
Lok, C.-N., E. Viazovkina, et al. (2002). Biochemistry 41(10): 3457-3467.
Mangos, M. M. and M. J. Damha (2002). Curr. Top. Med. Chem. 2(10): 1147-1171.
Mangos, M. M., K.-L. Min, et al. (2003). J. Am. Chem. Soc. 125(3): 654-661.
Naka, T., N. Minakawa, et al. (2000). J. Am. Chem. Soc. 122(30): 7233-7243.
Nielsen et al. (1991), Science 254:1497.
Novac, O., et al (2004), Nucleic Acids Res., 32, 902-915.
Pillai, R. S., et al. (2005), Science, 309, 1573-1576
Satoh, H., Y. Yoshimura, et al. (1998). Bioorg. Med. Chem. Lett. 8(9): 989-992.
Stephenson, M. L. and P. C. Zamecnik (1978). Proc. Natl. Acad. Sci. U.S.A. 75(1): 285-8.
Thibaudeau, C., J. Plavec, et al. (1998). J. Org. Chem. 63(15): 4967-4984.
Trempe, J.-F., C. J. Wilds, et al. (2001). J. Am. Chem. Soc. 123(21): 4896-4903.
Uhlmann, E. and A. Peyman (1990). Chem. Rev. 90(4): 543-84.
Wincott, F. E. (2000). Current Protocols in Nucleic Acid Chemistry Unit 3.5: 3.5.1-3.5.12.
Wu, H., W. F. Lima, et al. (1999). J. Biol. Chem. 274(40): 28270-28278.
Yoshimura, Y., M. Endo, et al. (1999). J. Org. Chem. 64 (21): 7912-7920.
Yoshimura, Y., K. Kitano, et al. (2000). Bioorg. Med. Chem. 8(7): 1545-1558.
Young, Robert J., S. Shaw-Ponter, et al. (1994). Tetrahedron Lett. 35(46): 8687-8690.
Yuasa, H., J. Tamura, et al. (1990). J. Chem. Soc., Perkin Trans. 1 (10): 2763-2769.

1. An oligonucleotide comprising at least one 4′-thioarabinose-modified nucleotide.

2. The oligonucleotide of claim 1, wherein the oligonucleotide is 5-100 nucleotides in length.

3. The oligonucleotide of claim 1, wherein the oligonucleotide further comprises one or more DNA-like nucleotides.

4. The oligonucleotide of claim 1, wherein the oligonucleotide further comprises one or more RNA-like nucleotides other than a 4′-thioarabinose-modified nucleotide.

5. The oligonucleotide of claim 3, wherein the oligonucleotide is capable of inducing RNase H-mediated cleavage of a complementary RNA strand.

6. The oligonucleotide of claim 1, wherein the oligonucleotide is 5′-phosphorylated.

7. The oligonucleotide of claim 2, wherein the oligonucleotide is capable of hybridizing to a complementary oligonucleotide thereby to form a double-stranded siRNA-like molecule, wherein the 4′-thioarabinose-modified nucleotide is present in either or both strands.

8. The oligonucleotide of claim 7, where one or both strands of the double-stranded siRNA-like molecule have overhangs from 1-5 nucleotides on the 3′-end.

9. (canceled)

10. The oligonucleotide of claim 8, wherein the overhanging nucleotides are DNA-like nucleotides.

11. The oligonucleotide of claim 10 wherein the DNA-like nucleotides are 2′-deoxyribonucleotides, 2′-deoxy-2′-fluoroarabinonucleotides or combinations thereof.

12. The oligonucleotide of claim 7, wherein neither strand has an overhang.

13. The oligonucleotide of claim 7, wherein the sense strand comprises a chemical modification at one or more terminal nucleotides, the modification conferring resistance to phosphorylation.

14. (canceled)

15. The oligonucleotide of claim 1, wherein the oligonucleotide is 15-80 nucleotides in length and comprises a first sequence and a second sequence complementary to said first sequence such that the oligonucleotide or a portion thereof is capable of adopting an siRNA-like hairpin structure in which the first and second sequences form the stem of the hairpin structure.

16. The oligonucleotide of claim 1, wherein the 4′-thioarabinose-modified nucleotide is present within the 5′-terminal 8 nucleotides of the oligonucleotide.

17. The oligonucleotide of claim 7, wherein the 4′-thioarabinose-modified nucleotide is present within the 5′-terminal 8 nucleotides of either or both strands of the double-stranded siRNA-like molecule.

18-19. (canceled)

20. The oligonucleotide of claim 17, wherein the 4′-thioarabinose-modified nucleotide is present within the 3′-terminal 8 nucleotides of the sense strand of the double-stranded siRNA-like molecule.

21-22. (canceled)

23. The oligonucleotide of claim 17, wherein one strand of the double-stranded siRNA-like molecule comprises the 4′-thioarabinose-modified nucleotide and the other strand comprises a 2′-deoxy-2′-fluoroarabinonucleotide.

24. The oligonucleotide of claim 23, wherein the strand comprising the 4′-thioarabinose-modified nucleotide is the antisense strand of the double-stranded siRNA-like molecule.

25. The oligonucleotide of claim 1, wherein the arabinose modified nucleotide comprises a 2′ substituent selected from the group consisting of fluorine, hydroxyl, amino, azido, alkyl, alkoxy, and alkoxyalkyl groups.

26-30. (canceled)

31. The oligonucleotide of claim 1, wherein the at least one 4′-thioarabinose modified nucleotide is a 2′-deoxy-2′-fluoro-4′-thioarabinonucleotide (2′F-4′S-ANA).

32. The oligonucleotide of claim 1, wherein the oligonucleotide comprises two or more types of arabinose-modified nucleotides.

33. The oligonucleotide of claim 7, wherein the two or more types of arabinose-modified nucleotides are present in the same strand, different strands or both strands of the double-stranded siRNA-like molecule.

34. The oligonucleotide of claim 32, wherein the two or more types of arabinose modified nucleotides are 2′-deoxy-2′-fluoro-4′-thioarabinonucleotide (2′F-4′S-ANA) and 2′-deoxy-2′-fluoro-arabinonucleotide (2′F-ANA).

35-39. (canceled)

40. An siRNA or siRNA-like molecule comprising the oligonucleotide of claim 1.

41. A double-stranded siRNA or siRNA-like molecule comprising (a) a first oligonucleotide comprising the oligonucleotide of claim 1 and (b) a second oligonucleotide complementary thereto.

42. The double-stranded siRNA or siRNA-like molecule of claim 41, wherein the second oligonucleotide comprises an oligonucleotide of comprising at least one 4′-thioarabinose-modified nucleotide.

43. The double-stranded siRNA or siRNA-like molecule according to claim 41, wherein the first and second oligonucleotides are 19 to 23 nucleotides in length.

44. The double-stranded siRNA or siRNA-like molecule of claim 41, wherein the double-stranded siRNA or siRNA-like molecule comprises a 19-21 bp duplex portion.

45. The double-stranded siRNA or siRNA-like molecule of claim 41, wherein the double-stranded siRNA or siRNA-like molecule comprises a 1-5 nucleotide 3′ overhang in one or both strands.

46. (canceled)

47. A method for increasing (a) therapeutic efficacy, (b) nuclease stability, (c) selectivity of binding or (d) any combination of (a) to (c), of an oligonucleotide, the method comprising:
(i) replacing at least one nucleotide of the oligonucleotide with a 4′-thioarabinose modified nucleotide;
(ii) incorporating a 4′-thioarabinose modified nucleotide into the oligonucleotide; or
(iii) both (i) and (ii).

48. The method of claim 47, wherein the 4′-thioarabinose modified nucleotide is a 2′-deoxy-2′-fluoro-4′-thioarabinonucleotide (2′F-4′S-ANA).

49. A composition comprising the oligonucleotide of claim 1 and a pharmaceutically acceptable carrier.

50-52. (canceled)

53. A method of inhibiting expression of a nucleic acid sequence or gene in a biological system, comprising introducing into the system the oligonucleotide claim 1 wherein the oligonucleotide is targeted to the nucleic acid sequence or gene.

54. A method of inhibiting expression of a nucleic acid sequence or gene in a subject, comprising administering a therapeutically effective amount of the oligonucleotide claim 1 to the subject, wherein the oligonucleotide is targeted to the nucleic acid sequence or gene.

55. A method of treating a condition associated with expression of a nucleic acid sequence or gene in a subject, the method comprising administering the oligonucleotide of claim 1 to the subject, wherein the oligonucleotide is targeted to the nucleic acid sequence or gene.

56. (canceled)

57. A method of preparing the oligonucleotide of claim 1, said method comprising incorporating at least one 4′-thioarabinose-modified nucleotide monomer during oligonucleotide synthesis.

58. A compound of the Formula I: 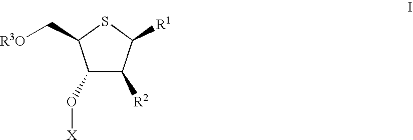
wherein:
R¹ is a canonical or modified nucleobase;
R² is selected from the group consisting of a halogen, OH, and alkoxy;
R³ is a protecting group; and
X is selected from the group consisting of a phosphoramidite moiety, an H-phosphonate moiety and a linker moiety capable of attachment to a solid support.

59. The compound of claim 58, wherein R² is a halogen selected from the group consisting of F and Cl.

60. The compound of claim 58, wherein R² is OMe.

61. The compound of claim 58, wherein the protecting group is selected from the group consisting of monomethoxytrityl, dimethoxytrityl, levulinyl, and silyl-based protecting groups.

62. The compound of claim 58, wherein X is a phosphoramidite moiety of the Formula II: 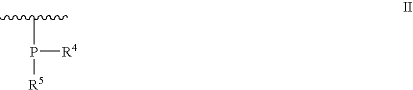
wherein:
R⁴ is a dialkylamino group NR⁹R¹⁰, wherein R⁹ and R¹⁰ are each independently lower alkyl groups, linear or branched; and
R⁵ is a substituted or unsubstituted alkoxy group OR¹¹, wherein R¹¹ is selected from the group consisting of methyl, beta-cyanoethyl, p-nitro-phenylethyl, trimethylsilylethyl, S-acetylthioethyl (AcS—CH₂CH₂—), or other lower alkyl, linear or branched, including substituted alkyl groups.

63. The compound of claim 58, wherein X is an H-phosphonate moiety of the Formula IV: 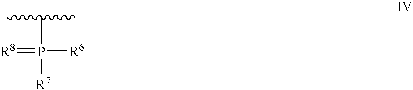
wherein:
R⁶ is H;
R⁷ is selected from the group consisting of OH and an oxyanion (O—) paired with a cationic ion; and
R⁸ is selected from the group consisting of O and S.

64. The compound of claim 58 of the Formula VI: 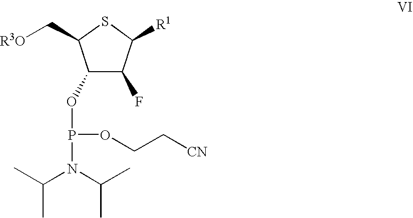
or a salt thereof.

65. A method of preparing the compound of claim 58, the method comprising:
(a) providing a compound of the Formula VIII: 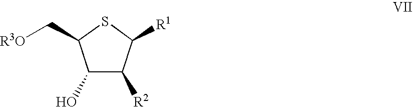
wherein R¹, R² and R³ are as defined in claim 58, and wherein if R¹ is a base selected from the group consisting of adenine, guanine and cytosine, the amino group thereof is masked by a protecting group; and
(b) phosphitylation of the 3′-hydroxyl group of the compound of (a).

66-67. (canceled)

68. A method of synthesizing the oligonucleotide of claim 1, the method comprising:
a. 5′-deblocking;
b. coupling;
c. capping; and
d. oxidation;
wherein (a), (b), (c) and (d) are repeated under conditions suitable for the synthesis of the oligonucleotide, and wherein the synthesis is carried out in the presence of a phosphoramidite or H-phosphonate monomer base comprising a compound of the Formula I: 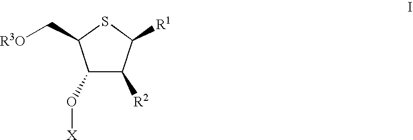
wherein:
R¹ is a canonical or modified nucleobase;
R² is selected from the group consisting of a halogen, OH, and alkoxy;
R³ is a protecting group; and
X is selected from the group consisting of a phosphoramidite moiety, an H-phosphonate moiety and a linker moiety capable of attachment to a solid support.

69-73. (canceled)

74. A composition comprising the siRNA or siRNA-like molecule of claim 40 and a pharmaceutically acceptable carrier.

75. A method of inhibiting expression of a nucleic acid sequence or gene in a biological system, comprising introducing into the system the siRNA or siRNA-like molecule of claim 40, wherein the oligonucleotide is targeted to the nucleic acid sequence or gene.

76. A method of inhibiting expression of a nucleic acid sequence or gene in a subject, comprising administering a therapeutically effective amount of the siRNA or siRNA-like molecule of claim 40 to the subject, wherein the oligonucleotide is targeted to the nucleic acid sequence or gene.

77. A method of treating a condition associated with expression of a nucleic acid sequence or gene in a subject, the method comprising administering the siRNA or siRNA-like molecule of claim 40 to the subject, wherein the oligonucleotide is targeted to the nucleic acid sequence or gene.
